(19) United States
(12) Patent Application Publication
(10) Pub. No.: US 2011/0082080 A1
(43) Pub. Date: Apr. 7, 2011

(54) COMPOSITIONS AND METHODS OF USING THE HUMAN PROISLET PEPTIDE RECEPTOR

(75) Inventor: Claresa S. Levetan, Rosemont, PA (US)

(73) Assignee: CUREDM GROUP HOLDINGS, LLC, Wilmington, DE (US)

(21) Appl. No.: 12/682,631

(22) PCT Filed: Oct. 10, 2008
(86) PCT No.: PCT/US08/79602

§ 371 Date: Dec. 9, 2010

Related U.S. Application Data
(63) Continuation-in-part of application No. 61/031,451, filed on Feb. 26, 2008.
(60) Provisional application No. 60/979,569, filed on Oct. 12, 2007.

(51) Int. Cl. A61K 38/10; C07K 2/00; C07K 7/08; G01N 33/53; A61K 38/00; A61P 3/10; A61P 3/06; A61P 9/00; A61P 9/10

(52) U.S. Cl. 514/7.3; 530/300; 530/326; 436/501; 514/6.9; 514/7.4; 514/20.9; 514/21.5

(57) ABSTRACT

Methods and compositions related to the use of Human proIslet Peptide Receptor (HIP) are disclosed herein. Compositions include peptides and peptidomimetics capable of binding the HIP receptors. Methods include screening assays for ligands of receptors and proteins involved in islet cell signaling.

### CROSS-REFERENCE TO RELATED APPLICATIONS

This application claims the benefit of U.S. Provisional Application No. 60/979,569, filed Oct. 12, 2007 and U.S. Provisional Application No. 61/031,451, filed Feb. 26, 2008. Both applications are incorporated by reference herein in their entirety.

### SUMMARY

Embodiments of the present disclosure relate to the Human proIslet Peptide (HIP) receptor. Further embodiments of the present disclosure relate to methods and assays for rational drug design of agents that can, like the HIP peptides, bind to the HIP receptor, thereby stimulating islet neogenesis and treating type 1 and type 2 diabetes and other pathologies associated with aberrant glucose metabolism.

Embodiments of the present disclosure relate to methods of screening for a compound that binds a HIP receptor and, further, screening for a compound that modulates islet neogenesis or is capable of modulating glucose-related diseases, including, but not limited to type 1 diabetes, type 2 diabetes and other pathologies associated with aberrant glucose metabolism.

Embodiments of the present disclosure also relate to agents that modulate HIP receptor activity. Further embodiments of the present disclosure relate to agents that bind to the HIP receptor, including peptides and peptidomimetics.

Embodiments of the present disclosure also relate to peptidomimetics of mediating proteins, including, but not limited to islet cell growth modulators such as, Reg1a, syndecan 2, fibronectin 1, annexin A3, PAX-1 (paired box protein), PDX-1 (pancreas duodenium homeobox 1) and NGN3 (neurogenenin 3) and ligands thereof.

Further embodiments of the present disclosure relate to methods of stimulating islet neogenesis, and treat type 1 and type 2 diabetes and other pathologies associated with aberrant glucose metabolism comprising administering mediating proteins, including, but not limited to, Reg 1a, syndecan 2, fibronectin 1, annexin A3, PAX-1 (paired box protein), PDX-1 (pancreas duodenium homeobox 1) and NGN3 (neurogenenin 3) , or peptidomimetics thereof.

### DESCRIPTION OF DRAWINGS

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of embodiments disclosed herein, reference should be had to the following detailed description taken in connection with the accompanying drawings, in which:

FIG. 1 is the three-dimensional structure of the human EXTL3 gene.

FIG. 2 is a graph depicting the insulin levels after incubation in culture with human pancreatic ductal tissue with HIP1, HIP2 and HIP3.

FIG. 3 is a graph depicting insulin levels in human pancreatic islet cultures after incubation with HIP1, HIP2, HIP3, hamster INGAP.

FIG. 4 is a graph depicting the insulin requirements in mice rendered diabetic with streptozotocin and treated with HIP1, HIP2, HIP3 and hamster INGAP.

FIG. 5 is a graph depicting the rate of fall in insulin requirements between placebo-treated and streptozotocin-treated mice rendered diabetic and HIP-treated mice, showing a significantly faster decline in insulin requirements among HIP-treated mice compared to control mice (p=0.004).

FIG. 6 depicts the increased islet mass and islet numbers identified by histological evaluation and insulin staining. HIP2 and HIP3 had significantly greater total islet mass (p=0.05) and significantly increased total islet numbers (p=0.022). Repeated study among streptozotcin-treated mice rendered diabetic with HIP2B (Optimized HIP) demonstrated a three-fold rise in the total number of islets compared to placebo-treated mice. Differences between the two groups of HIP2B and placebo were statistically significant for both an increase in total islet mass and total islet numbers, with the HIP2B group indicating larger values for both islet mass (M=416714.67, SD=121,389.01) and islet number (M=94, SD=32.74) than for the placebo group (islet mass: M=127410.67, SD=96930.78, islet number: M=31.67, SD=15.28).

The islet sizes between the HIP2B/Optimized HIP treated diabetic mice and placebo-treated mice, were not statistically significant on the dependent variable of islet size (t=0.708, p=0.518) indicating that normally sized islets were generated by HIP2B/Optimized HIP. Mann-Whitney U tests confirmed the finding of the t-tests.

FIG. 7 is an immunofluorescent stain for insulin on mouse pancreatic tissue treated with HIP.

FIG. 8A is a Western Blot analysis demonstrating human insulin expression from PANC-1 cells under non-reducing and reducing conditions in response to incubation with various HIP and Optimized HIPs. FIG. 8B are Ponceau Stains under non-reducing and reducing conditions in response to incubation with various HIP and Optimized HIPs.

FIG. 9A demonstrates PANC-1 cells treated with HIP2, and Optimized HIP peptides for four days, with pictures taken on day 7 at 200× magnification. FIG. 9B demonstrates the progression of PANC-1 cell morphology changes through 7 days (control, HIP2 and HIP2B), with pictures taken on days 1, 2, 3, 5 and 7 at 200× magnification. FIG. 9C demonstrates progression morphological changes of PANC-1 cells treated with control and Optimized HIPs (HIP2 Dimer and HIP2 PEG).

FIG. 10 is a stain for CK19 and DAP1 to show nuclei and insulin in human pancreatic cells following administration of HIP2B.

FIG. 11 is graph depicting glucose levels of three NOD mice after treatment with placebo and lysophylline (LSF), HIP 2 and LSF, and HIP2B and LSF.

FIG. 12 depicts the translocation of the HIP receptor after stimulation with HIP and Optimized HIP. A Cy3 double antibody immunohistochemical staining of PANC-1 cells was performed after treatment with 150 μM HIP and Optimized HIP peptides for 48 hours, demonstrating the translocation of the HIP receptor from the cell membrane of PANC-1 cells to the cytoplasm upon stimulation with HIP and Optimized HIP.

FIG. 13 demonstrates exposure adjusted PANC-1 cells in SFM and TSFM with HIP and Optimized HIP2B peptides. FIG. 14 demonstrates Western blot analyses of HIP2 and HIP2B enhancing EXTL3 translocation from the cytoplasmic membrane to the nucleus in human PANC-1 cells.

FIG. 15 demonstrates the immunofluorescent analyses of translocation of EXTL3 into the nucleus with HIP2 and Optimized HIP2B.

FIG. 16 demonstrates that Reg1a is expressed specifically in pancreas tissue.

FIG. 17 demonstrates that Reg3a is expressed specifically in pancreas tissue.

FIG. 18 demonstrates the analogous amino acid sequences between Reg 1a and Reg 3a.

FIG. 19 is the three-dimensional structure of the Reg 1a (A) and the Reg 3a (B) proteins. FIG. 19(c) highlights the bioactive HIP peptide sequence within the Reg 3a protein.

FIG. 20 demonstrates a beta cell proliferation and islet differentiation map in accordance with embodiments of the present disclosure.

FIG. 21 is a further refined map of HIP interactors in accordance with embodiments of the present disclosure.

FIG. 22 demonstrates HIP interactors localized at the cell membrane with protein interactors in the intracellular and extracellular environment in accordance with embodiments of the present disclosure.

FIG. 23 is a proposed pathway of islet neogenesis in humans. Reg 3a is hypothesized to be the rate-limiting factor in this pathway and is secreted within the pancreas. Binding of the HIP region of Reg 3a initiates the interaction between HIP and EXTL3, leading to translocation of EXTL3 from the cytoplasmic membrane to the nucleus. This, in turn, triggers signaling leading to differentiation of the progenitor into the four endocrine cell types required for islet neogenesis. SDC2 and FN1 may participate in the process of islet genesis by aiding in the signaling of progenitor cell differentiation and ANXA3 may promote angiogenesis and innervation of developing islets.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

It must also be noted that as used herein and in the appended claims, the singular forms “a”, “an”, and “the” include plural reference unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 50% means in the range of 45%-55%.

As used herein, “HIP” refers to a Human prolslet Peptide, including HIP1, HIP2, or HIP3.

As used herein, “optimized HIP” refers to variations of HIP, HIP1 and/or HIP2 wherein the peptide has been modified to increase the stability, solubility or bioavailability of HIP, HIP1 or HIP2 as described in the disclosure. Stability refers to the peptide's resistance to degradation by in-serum proteases which target and degrade non-Optimized HIP3, HIP1 and/or HIP2. Bioavailability refers to the amount of peptide available for in vivo therapeutic use by the target cells, pathways and/or systemic mechanisms based on the peptide's ability to avoid degradation by proteases and other systemic pathways that degraded non-Optimized HIP3, HIP1 and/or HIP2. In certain embodiments, Optimized HIP refers to HIP3, HIP1 and/or HIP2 that are blocked by the addition of an N-terminal acetyl group and a C-terminal amide group, pegylated, and a combination thereof.

As used herein, “treating” a condition or patient refers to taking steps to obtain beneficial or desired results, including clinical results. For purposes of this disclosure, beneficial or desired clinical results include, but are not limited to, alleviation or amelioration of one or more symptoms of diabetes, diminishment of extent of disease, delay or slowing of disease progression, amelioration, palliation or stabilization of the disease state, and other beneficial results described below. Symptoms of diabetes include low or inadequate levels of insulin or insulin activity, frequent urination, excessive thirst, extreme hunger, unusual weight loss, increased fatigue, irritability, blurry vision, genital itching, odd aches and pains, dry mouth, dry or itchy skin, impotence, vaginal yeast infections, poor healing of cuts and scrapes, excessive or unusual infections, hyperglycemia, loss of glycemic control, fluctuations in postprandial blood glucose, fluctuations in blood glucagon, fluctuations in blood triglycerides and include reduction in rate of or diminution of or improved outcomes with conditions that are accelerated by and/or occur because of or more frequently with earlier diabetes including macrovascular and microvascular disease inclusive but limited cerebrovascular impairment with or without stroke, angina, coronary heart disease, myocardial infarction, peripheral vascular disease, nephropathy, kidney impairment, increased proteinuria, retinopathy, neovascularization of vessels in the retina, neuropathy including central, autonomic and peripheral neuropathy that may lead to loss of sensation of extremities and amputation and/or from neuropathy or diminished vascular flow, skin conditions including but not limited to diabetic dermopathy, Necrobiosis Lipoidica Diabeticorum, bullosis diabeticorum, scleroderma diabeticorum, granuloma annulare bacterial skin Infections, but limited to Staphylococcus, which can result in deeper infections. Diabetes may be diagnosed by methods well known to one of ordinary skill in the art. For example, commonly, diabetics have a plasma blood glucose result of greater than 126 mg/dL of glucose. Prediabetes, which may also be treated by the compositions and methods of the disclosure, is commonly diagnosed in patients with a blood glucose level between 100 and 125 mg/dL of glucose. Other symptoms may also be used to diagnose diabetes, related diseases and conditions, and diseases and conditions affected by diminished pancreatic function.

As used herein, “reduction” of a symptom or symptoms (and grammatical equivalents of this phrase) means decreasing of the severity or frequency of the symptom(s), or elimination of the symptom(s).

The term “inhibiting” includes the administration of a compound of the present disclosure to prevent the onset of the symptoms, alleviating the symptoms, or eliminating the disease, condition or disorder.

As used herein, a “pathology associated with impaired pancreatic function” is one in which the pathology is associated with a diminished capacity in a subject for the pancreas of the subject to produce and/or secrete hormones and/or peptides. In certain embodiments, this hormone or cytokine is insulin. Pathologies that are associated with impaired pancreatic function include type 1 diabetes, new onset type 1 diabetes, type 2 diabetes, latent autoimmune diabetes of adulthood, pre-diabetes, impaired fasting glucose, impaired glucose tolerance, fasting hyperglycemia, insulin resistant syndrome, hyperglycemic conditions generally in children or adults and those with a family history of diabetes exhibiting an abnormal fasting glucose or insulin levels, metabolic syndrome, overweight, obesity, hyperlipidemia, cholesterolemia, hypertriglyceridemia, eating disorders, polycystic ovarian syndrome, anovulatory cycles, fasting hyperlipidemia/hypercholesterolemia, elevated fasting total cholesterol, elevated LDL and VLDL cholesterol, family history of diabetes and forms of impotence or sexual dysfunction associated with such conditions. “New onset” or “newly diagnosed” is defined as having been diagnosed with diabetes within the past 3 months, whereas “pre-existing” is defined as having been diagnosed with diabetes 3 months ago or longer.

As used herein, “administering” or “administration of” a drug or therapeutic to a subject (and grammatical equivalents of this phrase) includes both direct administration, including self-administration, directly into or onto a target tissue or to administer a therapeutic to a subject whereby the therapeutic positively impacts the tissue to which it is targeted, and indirect administration, including the act of prescribing a drug. For example, as used herein, a physician who instructs a patient to self-administer a drug and/or provides a patient with a prescription for a drug is administering the drug to the patient.

As used herein, a “subject” or “patient” is a mammal, typically a human, but optionally a mammalian animal of veterinary importance, including but not limited to horses, cattle, sheep, dogs, and cats.

As used herein, a “manifestation” of a disease refers to a symptom, sign, anatomical state (e.g., lack of islet cells), physiological state (e.g., glucose level), or report (e.g., triglyceride level) characteristic of a subject with the disease.

As used herein, a “therapeutically effective amount” of a drug or agent is an amount of a drug or agent that, when administered to a subject with a disease or condition will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations. Adminsitration may be by any means known in the art such as intravenous, intra-muscularly, orally, or otherwise.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

By “pharmaceutically acceptable”, it is meant the carrier, diluent or excipient must be compatible with the other ingredients of the formulation and not deleterious to the recipient thereof.

As used herein, “TID”, “QD” and “QHS” have their ordinary meanings of “three times a day”, “once daily,” and “once before bedtime”, respectively.

Administration of an agent “in combination with” includes parallel administration (administration of both the agents to the patient over a period-of time, such as administration of a monoclonal antibody and a peptide hormone such as an incretin hormone or analog on alternate days for one month), co-administration (in which the agents are administered at approximately the same time, e.g., within about a few minutes to a few hours of one another), and co-formulation (in which the agents are combined or compounded into a single dosage form suitable for oral, subcutaneous or parenteral administration).

“Hamster INGAP” is a non-human islet neogenesis associated peptide having the sequence Ile-Gly-Leu-His-Asp-Pro-Ser-His-Gly-Thr-Leu-Pro-Asn-Gly-Ser (SEQ ID NO: 1).

“HIP3” (Ile-Gly-Leu-His-Asp-Pro-Thr-Gln-Gly-Thr-Glu-Pro-Asn-Gly-Glu (SEQ ID NO: 2)) is a Human proIslet Peptide in purified, synthetic, or recombinant form. HIP3 has a molecular weight of about 1564.6.

“HIP1” (Trp-Ile-Gly-Leu-His-Asp-Pro-Thr-Gln-Gly-Thr-Glu-Pro-Asn-Gly (SEQ ID NO: 3)) is a Human proIslet Peptide in purified, synthetic, or recombinant form.

“HIP2” (Ile-Gly-Leu-His-Asp-Pro-Thr-Gln-Gly-Thr-Glu-Pro-Asn-Gly (SEQ ID NO: 4)) is a Human proIslet Peptide in purified, synthetic, or recombinant form. HIP2 has a molecular weight of about 1435.5.

HIP3Blocked or HIP3B (Ac-Ile-Gly-Leu-His-Asp-Pro-Thr-Gln-Gly-Thr-Glu-Pro-Asn-Gly-Glu-NH2) (SEQ ID NO: 5)) is a Human proIslet Peptide which has been blocked with a n-terminal acetyl group and a c-terminal amide group, in purified, synthetic, or recombinant form. HIPB has a molecular weight of about 1605.7.

HIP1 Blocked (Ac-Trp-Ile-Gly-Leu-His-Asp-Pro-Thr-Gln-Gly-Thr-Glu-Pro-Asn-Gly-NH2 (SEQ ID NO: 6)) is a Human proIslet Peptide which has been blocked with a n-terminal acetyl group and a c-terminal amide group, in purified, synthetic, or recombinant form.

HIP2Blocked or HIP2B (Ac-Ile-Gly-Leu-His-Asp-Pro-Thr-Gln-Gly-Thr-Glu-Pro-Asn-Gly-NH2) (SEQ ID NO: 7)) is a Human proIslet Peptide which has been blocked with a n-terminal acetyl group and an c-terminal amide group, in purified, synthetic, or recombinant form. HIP2B has a molecular weight of about 1476.6.

HIP3Cys (Ile-Gly-Leu-His-Asp-Pro-Thr-Gln-Gly-Thr-Glu-Pro-Asn-Gly-Glu-Cys) (SEQ ID NO: 8)) is a Human proIslet Peptide which has an additional c-terminal cysteine residue, in purified, synthetic or recombinant form.

HIP1 Cys (Trp-Ile-Gly-Leu-His-Asp-Pro-Thr-Gln-Gly-Thr-Glu-Pro-Asn-Gly-Cys) (SEQ ID NO: 9)) is a Human proIslet Peptide which has an additional c-terminal cysteine residue, in purified, synthetic or recombinant form.

HIP2Cys (Ile-Gly-Leu-His-Asp-Pro-Thr-Gln-Gly-Thr-Glu-Pro-Asn-Gly-Cys) (SEQ ID NO: 10)) is a Human proIslet Peptide which has an additional c-terminal cysteine residue, in purified, synthetic or recombinant form.

HIP3CysDimer (Ile-Gly-Leu-His-Asp-Pro-Thr-Gln-Gly-Thr-Glu-Pro-Asn-Gly-Cys-Cys-Gly-Asn-Pro-Glu-Thr-Gly-Gln-Thr-Pro-Asp-His-Leu-Gly-Ile) (SEQ ID NO: 11)) is a Human proIslet Peptide dimer wherein each monomer has been modified to include an c-terminal cysteine residue, in purified, synthetic, or recombinant form. The dimer forms via the creation of a disulfide bond between the cysteine residues of the individual monomers, as shown below:

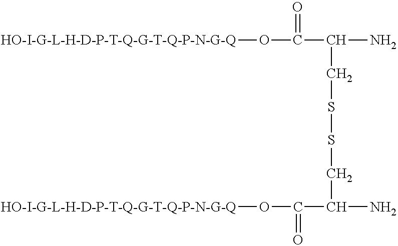

HIP1CysDimer (Trp-Ile-Gly-Leu-His-Asp-Pro-Thr-Gln-Gly-Thr-Glu-Pro-Asn-Gly-Glu-Cys-Cys-Glu-Gly-Asn-Pro-Glu-Thr-Gly-Gln-Thr-Pro-Asp-His-Leu-Gly-Ile-Trp) (SEQ ID NO: 12)) is a Human proIslet Peptide dimer wherein each monomer has been modified to include an c-terminal cysteine residue, in purified, synthetic, or recombinant form. The dimer forms via the creation of a disulfide bond between the cysteine residues of the individual monomers, as shown below:

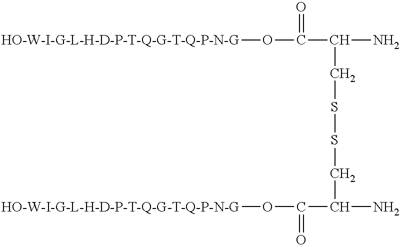

HIP2CysDimer (Ile-Gly-Leu-His-Asp-Pro-Thr-Gln-Gly-Thr-Glu-Pro-Asn-Gly-Cys-Cys-Gly-Asn-Pro-Glu-Thr-Gly-Gln-Thr-Pro-Asp-His-Leu-Gly-Ile) (SEQ ID NO: 13)) is a Human proIslet Peptide dimer wherein each monomer has been modified to include an c-terminal cysteine residue, in purified, synthetic, or recombinant form. The dimer forms via the creation of a disulfide bond between the cysteine residues of the individual monomers, as shown below:

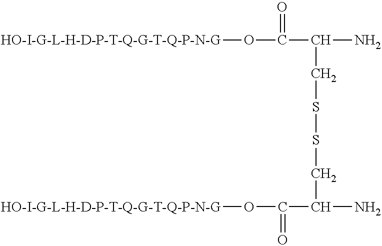

HIP3CysBlocked (Ac-Ile-Gly-Leu-His-Asp-Pro-Thr-Gln-Gly-Thr-Glu-Pro-Asn-Gly-Glu-Cys-NH2) (SEQ ID NO: 14)) is a Human proIslet Peptide which has been modified to include a c-terminal cysteine residue and has been blocked with a n-terminal acetyl group and a c-terminal amide group, in purified, synthetic, or recombinant form.

HIP1CysB locked (Ac-Trp-Ile-Gly-Leu-His-Asp-Pro-Thr-Gln-Gly-Thr-Glu-Pro-Asn-Gly-Cys-NH2) (SEQ ID NO: 15)) is a Human proIslet Peptide which has been modified to include a c-terminal cysteine residue and has been blocked with a n-terminal acetyl group and a c-terminal amide group, in purified, synthetic, or recombinant form.

HIP2CysBlocked (Ac-Ile-Gly-Leu-His-Asp-Pro-Thr-Gln-Gly-Thr-Glu-Pro-Asn-Gly-Cys-NH2) (SEQ ID NO: 16)) is a Human proIslet Peptide which has been modified to include a c-terminal cysteine residue and has been blocked with a n-terminal acetyl group and a c-terminal amide group, in purified, synthetic, or recombinant form.

HIP3CysBlockedDimer (2(Ac-Ile-Gly-Leu-His-Asp-Pro-Thr-Gln-Gly-Thr-Glu-Pro-Asn-Gly-Glu-Cys-NH2)) (SEQ ID NO: 17) is a Human proIslet Peptide dimer wherein each monomer has been modified to include a c-terminal cysteine residue and has been blocked with a n-terminal acetyl group and a c-terminal amide group, in purified, synthetic, or recombinant form. The dimer forms via the creation of a disulfide bond between the cysteine residues of the individual monomers, as shown below:

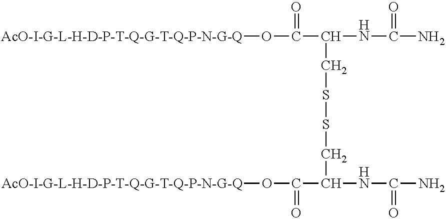

HIP1CysBlockedDimer (2(Ac-Trp-Ile-Gly-Leu-His-Asp-Pro-Thr-Gln-Gly-Thr-Glu-Pro-Asn-Gly-Cys-NH2)) (SEQ ID NO: 18) is a Human proIslet Peptide dimer wherein each monomer has been modified to include a c-terminal cysteine residue and has been blocked with a n-terminal acetyl group and a c-terminal amide group, in purified, synthetic, or recombinant form. The dimer forms via the creation of a disulfide bond between the cysteine residues of the individual monomers, as shown below:

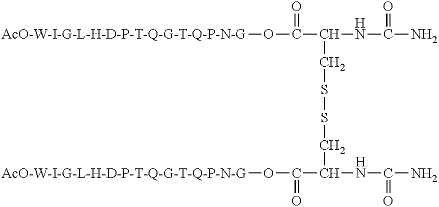

HIP2CysBlockedDimer or HIP2B Cys Dimer (2(Ac-Ile-Gly-Leu-His-Asp-Pro-Thr-Gln-Gly-Thr-Glu-Pro-Asn-Gly-Cys-NH2) (SEQ ID NO:19) is a Human proIslet Peptide dimer wherein each monomer has been modified to include a c-terminal cysteine residue and has been blocked with a n-terminal acetyl group and a c-terminal amide group, in purified, synthetic, or recombinant form. The dimer forms via the creation of a disulfide bond between the cysteine residues of the individual monomers, as shown below:

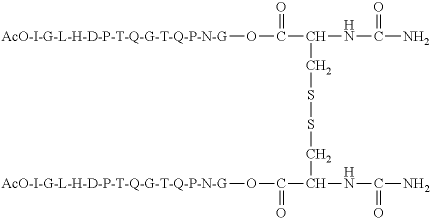

HIP2B Cys Dimer has a molecular weight of about 3157.5.

HIP3CysPEG (Ile-Gly-Leu-His-Asp-Pro-Thr-Gln-Gly-Thr-Glu-Pro-Asn-Gly-Glu-Cys-PEG) (SEQ ID NO: 20)) is a Human proIslet Peptide which has been modified to include a c-terminal cysteine residue to which has been covalently bonded to a dimeric maleimide activated 40 Kd PEG construct, in purified, synthetic, or recombinant form.

HIP1 CysPEG (Trp-Ile-Gly-Leu-His-Asp-Pro-Thr-Gln-Gly-Thr-Glu-Pro-Asn-Gly-Cys-PEG) (SEQ ID NO: 21)) is a Human proIslet Peptide which has been modified to include a c-terminal cysteine residue to which has been covalently bonded to a dimeric maleimide activated 40 Kd PEG construct, in purified, synthetic, or recombinant form.

HIP2CysPEG (Ile-Gly-Leu-His-Asp-Pro-Thr-Gln-Gly-Thr-Glu-Pro-Asn-Gly-Cys-PEG) (SEQ ID NO: 22) is a Human proIslet Peptide which has been modified to include a c-terminal cysteine residue to which has been covalently bonded to a dimeric maleimide activated 40 Kd PEG construct, in purified, synthetic, or recombinant form.

HIP3CysBlockedPEG (Ac-Ile-Gly-Leu-His-Asp-Pro-Thr-Gln-Gly-Thr-Glu-Pro-Asn-Gly-Glu-Cys-PEG-NH2) (SEQ ID NO: 23) is a Human proIslet Peptide which has been blocked with a n-terminal acetyl group and a c-terminal amide group, and modified to include a c-terminal cysteine residue to which has been covalently bonded to a dimeric maleimide activated 40 Kd PEG construct, in purified, synthetic, or recombinant form as shown below:

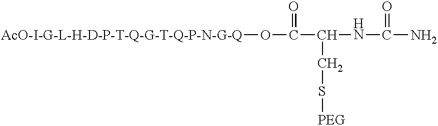

HIP1 CysBlockedPEG (Ac-Trp-Ile-Gly-Leu-His-Asp-Pro-Thr-Gln-Gly-Thr-Glu-Pro-Asn-Gly-Cys-PEG-NH2) (SEQ ID NO: 24) is a Human proIslet Peptide which has been blocked with a n-terminal acetyl group and an c-terminal amide group, and modified to include a c-terminal cysteine residue to which has been covalently bonded to a dimeric maleimide activated 40 Kd PEG construct, in purified, synthetic, or recombinant form.

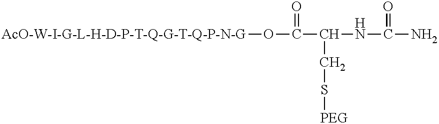

HIP2CysBlockedPEG or HIP2B Cys-PEG (Ac-Ile-Gly-Leu-His-Asp-Pro-Thr-Gln-Gly-Thr-Glu-Pro-Asn-Gly-Cys-PEG-NH2) (SEQ ID NO: 25) is a Human proIslet Peptide which has been blocked with a n-terminal acetyl group and a c-terminal amide group, and modified to include a c-terminal cysteine residue to which has been covalently bonded to a dimeric maleimide activated 40 Kd PEG construct, in purified, synthetic, or recombinant form. HIP2B Cys-PEG has a molecular weight of about 44,782.

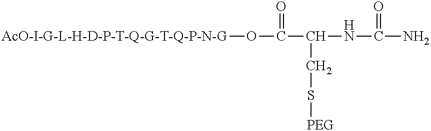

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, sequence variants thereof, as well as non-peptide agents such as small molecule drug mimetics, as further described below.

The term “peptide” refers to a short amino acid sequence. Such amino acids sequences can be less than 30 amino acids, less than 20 amino acids, less that 10 amino acids or smaller. In some embodiments the peptide sequence is 4, 5, 6, 7, 8, 9, 10, 11, 12, 13, 15, 16, 17, 18, 19, or 20 amino acids long and may range in size of any there between. Peptides can be produced by any means know in the art, including chemical synthesis, recombinant protein engineering, proteolytic digest or any other method. Such peptides described herein may be peptidomimetics.

The “percent identity” of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules encoding a peptide sequence as described herein. BLAST protein searches can be performed with the XBLAST program, score=50, wordlength=3 to obtain amino acid sequences homologous to a peptide as described herein. To obtain gapped alignments for comparison purposes, Gapped BLAST can be utilised as described in Altschul et al, Nucleic Acids Res. (1997) 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See, for example, http:www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

The skilled person is aware that various amino acids have similar properties. One or more such amino acids of a substance can often be substituted by one or more other such amino acids without eliminating a desired activity of that substance. Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). Substitutions of this nature are often referred to as “conservative” or “semi-conservative” amino acid substitutions.

Amino acid deletions or insertions may also be made relative to the amino acid sequence of a peptide sequence as described herein. Thus, for example, amino acids which do not have a substantial effect on the activity of such peptides, or at least which do not eliminate such activity, may be deleted. Amino acid insertions relative to the sequence of peptides as described herein can also be made. This may be done to alter the properties of a protein of the present invention (e.g. to assist in identification, purification or expression, where the protein is obtained from a recombinant source, including a fusion protein. Such amino acid changes relative to the sequence of a peptide from a recombinant source can be made using any suitable technique e.g. by using site-directed mutagenesis. The molecule may, of course, be prepared by standard chemical synthetic techniques, e.g. solid phase peptide synthesis, or by available biochemical techniques. Embodiments contemplated herein comprise amino acid sequences that are 80%, 85%, 90%, 95%, or greater in sequence identity to a reference sequence such as, for example, HIP, HIP2B, the HIP receptor, and any other amino acid sequence disclosed herein.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

Since 1922, insulin has been the only available therapy for the treatment of type 1 diabetes and other conditions related to lack of or diminished production of insulin. It is well established that at the onset of type 1 diabetes, patients have already lost at least 90% of their islets and their number of islets continues to steadily decline. However what has recently become clear is that not only in type 1 diabetes is there a deficit of islet mass, but also at the time of diagnosis of type 2 diabetes, patients exhibit a loss of at least 50% of the islet mass and number. As with type 1 patients, the number and mass of islets continues to decline in type 2 diabetes, not from autoimmune attack, but because the beta cells effectively become “burned out.” Although this decline occurs more rapidly in type 1 patients, there is still a decline of 10-20% per year among type 2 patients.

A common misunderstanding is that insulin resistance causes type 2 diabetes. Although insulin resistance is a feature of both diabetes and obesity, diabetes does not occur as a result of insulin resistance without the coexistence of reduction of islet mass leading to reduction in insulin secretion. Diabetes occurs only when there is a critical reduction in islet mass and function that prevents an adequate insulin response to a carbohydrate stimulus. Despite some regenerative ability of the beta cells within the islet structures, the islets have a much slower ability to regenerate.

Even in the face of blocking the autoimmune destruction of the insulin-producing cells in the pancreas, without new methods of regenerating islets, there will not be an end to type 1 diabetes.

Loss of islet mass is the basis of both type 1 and 2 diabetes, and more recent studies have demonstrated that prediabetes, insulin resistant states, hypertension, inactivity and family history are islet stressors with diminished islet mass.

Despite decades of research and the advent of pancreatic islet cell transplantation and newer claims of success resulting from the Edmonton Protocol for islet cell transplantation, the success has not been replicated in the United States. At four years post-transplant, fewer than 10% of patients who have received islet cell transplants remain insulin independent. Additionally, despite new immune suppression protocols, there is an 18% rate per patient of serious side effects.

In a normally functioning pancreas, small numbers of islets die naturally on a day-by-day basis and are replaced as required to keep glucose levels under control. On average, this regenerative process known as islet neogenesis replaces islets at a rate of approximately 2% per month. In nondiabetic patients, the beta cell mass within the existing islets can expand or contract depending on the insulin needs of the individual. This process is referred to as “beta cell proliferation” does not occur in patients with type 1 diabetes and is limited in type 2 patients.

The study of islet neogenesis is not new. In 1920, it was reported that an obstructive pancreatic stone resulted in atrophy to most of the pancreas but an increase in islets. It was then hypothesized that ligating (binding) the pancreatic ducts might lead to the identification of a substance that could be useful in the treatment of diabetes. Nearly a century ago, based upon autopsy findings from fatal pancreatic stones with the result being islet proliferation, surgeons in the early 1900s ligated the pancreatic tail of diabetic children in the hopes of producing substances that would form new islets. Although the positive effects of these procedures were short-lived, they demonstrated the potential for islet restoration in humans.

Pancreatic ligation studies that were intended to create a hamster model for pancreatitis resulted in the formation of many new islets. This research led to the isolation of a hamster peptide referred to as the Islet Neogenesis Associated Peptide, or INGAP. In the clinical development of INGAP, it was further demonstrated that new human islets could be differentiated from the stem-cell-like islet progenitor cells that reside throughout the adult pancreas even decades after the onset of type 1 diabetes.

Separate from the concept of using pancreatic ligation to produce new islets, regeneration of islets during pregnancy has been described. Islets are formed in late embryogenesis and pregnancy data demonstrates the islet population grows postnatally. Research has demonstrated that islet neogenesis precedes beta cell expansion during pregnancy. Furthermore, it has been described that postnatally, in humans, there are precursor cells within the pancreas, that are capable of expansion occur naturally and efficiently differentiate into clusters of islets.

The primary way in which patients with type 1 or later-stage type 2 diabetes manage their disease is by administering insulin, either via subcutaneous injection or by using a subcutaneous pump infusion. As well as the obvious lifestyle disadvantages, insulin therapy does not match the body's normal glucose control mechanisms and therefore does not fully manage glucose fluctuations. Even the best-controlled type 1 diabetic patients do not have anything remotely like a normal glucose metabolism. This is because insulin secretion is only part of the missing pancreatic function.

Over the past several decades, there have been several new therapies have become available for diabetes, which may improve metabolic function of the existing beta cells or islets within the pancreas. These are agents which may improve existing islet function and glucose metabolism include: Glucagon Like Peptide-1 (GLP-1) and its analogs, Gastric Inhibitory Peptide/Glucose-Dependent Insulinoptropic polypeptide (GIP), Amylin, and its analog, Pramlintide, and GLP-1 receptor agonists, such as Liraglutide (NN2211) and Exendin-4, or compounds which halt the destruction of GLP-1, such as Dipeptidyl Peptidase-4 Inhibitors, (DPP-4 inhibitors), and including but not limited to Vildagliptin, Sitagliptin, Saxagliptin , and PHX1149. Other compounds which may improve existing islet function include: gastrin, epidermal growth factor-1 and insulin sensitizing agents including the biguanide, Metformin, and the thiazolidinediones, Rosiglitazone and Pioglitazone. Other agents that may impact pancreatic function that may be utilized with the islet cell neogenesis agent include AGI-1067, an anti-inflammatory antioxidant agent that works by inhibiting signaling pathways that are activated in response to oxidative stress and pro-inflammatory stimuli, Rimonabant and other drugs that block the cannabinoid receptor 1 (CB 1), gut peptide, PYY, inclusive of, but not limited to PYY3-36 (PYY) nasal spray, the hypothalamic neuropeptide Y (NPY) and drugs that impact the leptin, ghrelin, pro-opiomelanocortin/melanocortin pathways or the melanocortin receptor, orlistat, sibutramine or acarbose.

Proof of the elasticity of the pancreas with respect to the generation of new pancreatic cells throughout one's lifetime accompanied by pancreatic cell death or apoptosis has replaced the long held concept that the number of insulin producing islet cells is fixed at birth and sustained throughout life. It is currently accepted that pancreatic islet cell neogenesis occurs from progenitor cells that exist within the adult pancreas. Studies confirm that progenitor cells exist within both the islet and ductal fractions of the adult human pancreas, and that upon stimulation with HIP, there is both increased insulin production along with islet numbers. This supports the data on pancreatic plasticity during pregnancy where studies among type 1 women, as many as ⅓ of women have a dramatic reduction in insulin requirements, with some women coming off insulin completely during their pregnancy. Even among patients who have had type 1 diabetes for decades, during pregnancy, many secrete normal levels of C-peptide, when C-peptide was non-detectable on the onset of pregnancy. Similarly, patients with type 1 diabetes having received renal transplants and on long term immunosuppression have been observed to regenerate insulin producing islet.

Additionally, over the past decade, clinical trials have been conducted to evaluate the impact of a number of immune modulators that may arrest the destruction of the pancreas. The studies and types of agents to potentially arrest the destruction of islet cells have varied considerably. The types of agents include general immunosuppressant agents which have typically been used in organ transplants, specifically targeted antibodies to those lymphocytes which attack the islets, along with other agents such as Vitamin D, in which a deficiency has been associated with a higher incidence of diabetes.

Anti CD-3 antibodies that target the immune response and specifically block the T-lymphocytes that cause islet cell death in type 1 diabetes have been utilized as well as heat-shock proteins to arrest the destruction of insulin-producing cells and anti-GAD65 antibody vaccines. Trials are underway with a number of diverse agents or combination of agents among newly diagnosed patients with diabetes. Currently the immune agents mycophenolate mofetil, Rituximab, an anti CD20 agent, which is an FDA approved agent for the treatment of B-lymphocyte lymphoma, is also being studied in the preservation of islet cells among newly diagnosed type I diabetes patients.

Trials are underway in newly diagnosed type I diabetes patients using the anti CD3 antibody, hOKT3 gammal (Ala-Ala), also known as MGA031 and the monoclonal antibody TRX4 (ChAglyCD3). The immune tolerance agent may also include, Polyclonal Anti-T-Lymphocyte Globulin (ATG), CTLA4-Ig (Abatacept) a selective costimulation modulator as it inhibits the costimulation of T cells, Campath-1H, (Anti-CD52 Antibody), a humanized monoclonal antibody to T-cells. Polyclonal Anti-T-Lymphocyte Globulin (ATG), DiaPep277, a derivative Heat Shock Protein 60, that may activate a subgroup of T-cells, which down-regulate T lymphocytes, anti-GAD antibody vaccine based on the 65 kDa isoform of the recombinant human glutamic acid decarboxylase protein (rhGAD65). DiaPep277 is another immune tolerance agent directed at the onset of type 1 diabetes to halt the destruction of islets. DiaPep277 is a heat shock protein, which is believed to impact the release of cytokines and pro-inflammatory cells which destroy islet cells, is being studied in adults and children with newly diagnosed patients with diabetes and also in patients with Latent Autoimmune Diabetes in Adults (LADA). CTLA4-Ig (Abatacept) inhibits a crucial stimulatory pathway in the activation of T cells. By this mechanism, the drug is thought to arrest or slow the T cell mediated autoimmune destruction of insulin producing cells and preserve their function. CTLA-4 Ig is being trialed as an intravenous agent begun within three months of diagnosis and then monthly for a total of 25 treatments. CampathHl is another immune tolerance agent being trialed among new onset type 1 diabetes and may be utilized in conjunction with HIP, Optimized HIP, hamster INGAP and other islet neogenesis agents for improvement in type 1 diabetes. Other agents under study, which inhibit attack on the pancreas, which will be used in conjuction with HIP2, Optimized HIPs to protect newly formed islets stimulated by HIP and optimized HIP include Granulocyte colony-stimulating factor, Neulasta (Pegfilgrastim), Vitamin D, both 25 hydroxy and 1,25 hydroxyvitamin D supplementation; IBC-VSO vaccine, which is a synthetic, metabolically inactive form of insulin designed to prevent pancreatic beta-cell destruction; interferon-alpha; a vaccine using CD4⁺CD25⁺ antigen-specific regulatory T cells or any agent or agents designed to suppress the immune attack upon beta cells within the islets of Langerhans, Prochymal (Human Adult Stem Cells), the anti-inflamatory Anakinra and the antinflamatory agent, Deoxyspergualin, an anti-inflamatory agent that blocks proinflammatory cytokine production and inhibits T-cells and B-cells, immune destruction of the islet cells while enhancing further transformation of new islets, which is a very slow process. Thus, immune therapy alone, even when delivered to newly diagnosed type 1 diabetes patients, has not been able to render patients insulin-free. Typically, a healthy individual requires about 1.5 million islet cells to maintain glucose homeostatsis. At the time of diagnosis, both type 1 and type 2 patients only retain about 50% or less of their typical islet cell mass (type 1 patients retain 10% or less of their insulin-producing cell function, while type 2 patients retain about 50% of their insulin-producing cell function). This ongoing destructive process in type 1 diabetes is typically more rapid and progressive than in type 2 diabetes leading to multiple daily insulin injections to survive. The typical healthy adult has an estimated cell death rate for islets of between 1000 and 2000 cells per day; the human islet lifespan is estimated at about 3 years. Each day, the same number of new islets are formed from precursor cells within the pancreas, both in the endocrine and exocrine portions of the organ. Thus, even if immune-halting agents are used to prevent further islet loss, because the daily regeneration rate of new islet production is only about 0.1% per day, it could take years, if not decades, to repopulate the pancreas with insulin producing cells without such an immune-blocking compound being combined with a regeneration compound such as Human proIslet Peptide (HIP), Optimized HIP, hamster INGAP or other islet neogenesis agents.

Embodiments of the present disclosure relate to a binding site for Human proIslet Peptide (HIP), and the role of this gene is the treatment of metabolic conditions such as type 1 and 2 diabetes. Embodiments of the present disclosure provide the HIP receptor along with the necessary proteins that also interact with the HIP receptor (and potentially act as islet cell growth modulators), which include: Reg1a, syndecan 2, fibronectin 1, annexin A3, PAX-1 (paired box protein), PDX-1 (pancreas duodenium homeobox 1) and NGN3 (neurogenenin 3). After interaction with of HIP with the HIP receptor, recruitment of Reg 1b, PDX-1, PAX-1, NGN3 by HIP further coordinates and regulates endocrine islet function and beta cell proliferation. Further embodiments of the present disclosure provide methods for the prevention and/or treatment of metabolic diseases including diabetes, which result from the interaction between HIP and its binding to the HIP receptor in the presence of necessary protein interactors. As used herein, the term “interactor” describes any protein, nucleotide or small molecule that interacts with a signaling molecule directly or indirectly. Such interaction may be receptor mediated, transient, or even mediated by other interactors in the receptor signally pathway.

Compounds identified using various methods of embodiments of the disclosure may be further tested for binding to the HIP receptor and/or to determine the compound's ability to stimulate or inhibit activity of the HIP receptor or modulate the activity of the HIP receptor by, for example, testing for HIP receptor activity, islet neogenesis or testing the candidate compound for binding to the HIP receptor. Such testing may be carried out by any method. For example, such methods may include contacting a known substrate with an identified compound and detecting binding to the HIP receptor by a change in fluorescence in a marker or by detecting the presence of the bound compound by isolating the HIP receptor/candidate compound complex and testing for the presence of the compound. In other embodiments, HIP receptor activity may be tested by, for example, isolating a substrate peptide that has or has not been phosphorylated by HIP or isolating a HIP receptor that has been contacted with the candidate compound. Such methods are well known in the art and may be carried out in vitro, in a cell-free assay, or in vivo, in a cell-culture assay.

Methods of testing binding and affinity are well known in the art. Specifically, a receptor and its cognate ligand bind to each other with a measurable affinity. Such affinity can be expressed using the formula:

$${K}_{d} = \frac{[ P ] [ L ]}{[ C ]}$$

The equilibrium dissociation constant is denoted K_{d} and is the inverse of the affinity constant Kₐ. In certain embodiments, the binding affinity (Ka) is very high such as between 0.1-10 nM. Those of ordinary skill in the art recognize that ligands may act as either an agonist or antagonist to the receptor, thereby activating or deactivating receptor signally, respectively. Moreover, certain ligands, particularly those with high affinities, may act as competitive inhibitors by displacing a lower affinity ligand, thereby altering receptor signaling. Such ligands are contemplated as embodiments of this disclosure. In certain embodiments, the agonist can act as an pancreatic islet cell growth modulator.

Embodiments of the present disclosure also relate to the Human proIslet Peptide (HIP) receptor. Further embodiments of the present disclosure relate to methods and assays for rational drug design of agents that can, like the HIP peptides, bind to the HIP receptor, thereby stimulating islet neogenesis and treating type 1 and type 2 diabetes and other pathologies associated with aberrant glucose metabolism. Such methods and assays may include comparing the binding of agents to known peptides that bind to the HIP receptor, including, but not limited to HIP1, HIP2, HIP3 and Optimized HIP. These peptides are known reagants that are useful in illustrating or validating the various embodiments of methods and assays of the present disclosure.

Embodiments of the present disclosure relate to methods of screening for a compound that binds a HIP receptor and, further, screening for a compound that modulates islet neogenesis or is capable of modulating glucose-related diseases, including, but not limited to type 1 diabetes, type 2 diabetes and other pathologies associated with aberrant glucose metabolism. Such methods include screening test compounds such as small organic molecule, peptides, nucleic acids and the like.

Embodiments of the present disclosure also relate to agents that modulate HIP receptor activity. Further embodiments of the present disclosure relate to agents that bind to the HIP receptor, including peptides and peptidomimetics.

Embodiments of the present disclosure also relate to peptidomimetics of mediating proteins, including, but not limited to, Reg 1a, syndecan 2, fibronectin 1, annexin A3, PAX-1 (paired box protein), PDX-1 (pancreas duodenium homeobox 1) and NGN3 (neurogenenin 3). After interaction with of HIP with the HIP receptor, recruitment of Reg 1b, PDX-1, PAX-1, NGN3 by HIP further coordinates and regulates endocrine islet function and beta cell proliferation.

Further embodiments of the present disclosure relate to methods of stimulating islet neogenesis, and treat type 1 and type 2 diabetes and other pathologies associated with aberrant glucose metabolism comprising administering mediating proteins, including, but not limited to, Reg 1a, syndecan 2, fibronectin 1, annexin A3, PAX-1 (paired box protein), PDX-1 (pancreas duodenium homeobox 1) and NGN3 (neurogenenin 3), or peptidomimetics thereof.

Human proIslet Peptide (HIP) is generally a bioactive portion of the Reg3A gene on chromosome 2p12. The HIP gene and peptide sequence are highly conserved among mammalian species with species variation from humans being single nucleotide polymorphisms. The HIP peptides provide for the transformation of progenitor cells in the adult human pancreas into new islet structures. The cell surface receptor protein interaction between HIP and the HIP receptor and the resultant cascade of interactions between the cell surface protein receptor and HIP leading to islet neogenesis is described herein.

HIP interacts with a cell surface receptor protein, which signals trafficking from the cell membrane to the nucleus resulting in islet neogenesis. This receptor protein, referred to herein as the HIP receptor, has been previously described, but its role in islet neogenesis has been previously unknown. This receptor has been categorized as falling under the group of what has been called the human exostoses-like proteins. The HIP protein receptor site in human pancreatic cells is the exostoses-like 3/EXTL3 protein and is a 919 amino acid protein located on chromosome 8p21. The HIP receptor protein, exostoses-like 3/EXTL3 has also been referred to as DKFZp686C2342, RPR, boty, REGR, botv, EXTR1 and KIAA0519. It has now been demonstrated that in order for islet neogenesis to occur in human pancreatic tissue, it would appear that HIP must bind to the HIP receptor in the presence of certain proteins, including, for example, Reg1a, syndecan 2, fibronectin 1, annexin A3, PAX-1 (paired box protein), PDX-1 (pancreas duodenium homeobox 1) and NGN3 (neurogenenin 3).

The EXTL3 protein was initially described as having similarities to the EXT family, but it was noted by the researchers that EXTL3 is not derived from the known EXT and EXTL genes. It was hypothesized that along with EXT1 and EXT2, these genes have tumor suppressor activity and that loss of function of these genes may contribute to the development of bone and breast tumors.

Embodiments of the present disclosure also provide four proteins as being mediating proteins that interact with the HIP receptor that appear to be involved in islet neogenesis when HIP binds to the HIP receptor in order for a signaling pathway to be initiated that is a cascade between the cell membrane and the nucleus resulting in islet neogenesis. These mediating proteins, which interact with HIP receptor for HIP to result in islet neogenesis include Reg1a, syndecan 2, fibronectin 1, annexin A3, PAX-1 (paired box protein), PDX-1 (pancreas duodenium homeobox 1) and NGN3 (neurogenenin 3). After interaction with of HIP with the HIP receptor, recruitment of Reg1b, PDX-1, PAX-1, NGN3 by HIP further coordinates and regulates endocrine islet function and beta cell proliferation. Reg1a is also referred to as pancreatic thread protein and regenerating islet-derived 1 alpha. Syndecan 2 is also called Sdc2, fibroglycan, heparan sulfate proteoglycan core protein, HSPG, Hspg1, Synd2, SYND2 and Syndecan-2 precursor. Fibronectin 1 is also known as FN1, FN, CIG, MSF, FINC, LETS, DKFZp686H0342, DKFZp68611370, DKFZp686F10164, DKFZp686O13149. Annexin A3 or ANXA3 and Hedgehog-interacting protein is also known as HHIP and Hedgehog-interating protein precursor.

EXTL3 has been considered to be part of a family of exocytosis-like proteins, although EXTL3 is considerably different both in its structure and function and whereas other proteins considered to be in the exocytosis-like protein family have been designated EXTL3 is considerably different from the other members of this family, and it is only EXTL3 that participates in the interaction with HIP and not other members of the family, including but not limited to EXT1, EXT2, EXTL1 and EXTL2. Whereas the HIP receptor, EXTL3, is an 919 amino acid protein and mapped on chromosome 8p21, EXT1 is a 746 amino and located on chromosome 1p21. EXTL2, also known as EXTR2, is 330 amino acid and resides on 1p11-p12.

The role and function the human protein EXTL3, which interacts with HIP and results in a cascade of protein to protein interactions leading to islet neogenesis in human pancreatic cells has previously been unknown. Animal models have noted the role of the protein receptor in beta cell regeneration. The findings presented herein, via molecular and protein mapping, demonstrate that beta cell regeneration/proliferation is a distinctly different process from islet neogenesis. Beta cell regeneration/explanation occurs within the beta cell compartment of existing islets, whereas it is demonstrated herein that the interaction between HIP and the HIP receptor protein results in islet neogenesis, not beta cell expansion/regeneration. These findings are similar to findings that demonstrate in utero, that islet neogenesis is a distinctly different process from beta cell regeneration/expansion. It is islet neogenesis that precedes the ability of the beta cells within islets to expand. Islet neogenesis is a far more comprehensive process than beta cell regeneration/expansion and the pathways leading to each have different protein to protein interactions.

Whereas some diabetes therapies are hypothesized to operate by increasing islet mass, the data disclosed herein suggest that compounds such as DPP4 inhibitors and GLP-1 receptor analogs may impact beta cell proliferation within existing islets rather than the formation of new islets.

As described herein, HIP interacts with an identified membrane bound protein receptor in the pancreas leading to a pancreas-specific cascade of protein interactions giving rise to islet neogenesis.

The reg proteins in humans include Reg 1a, Reg 1b, Reg 3a, Reg 3b, and Reg 4. Reg 2 has not been described in humans. Only Reg 1 and Reg 3 variants are clustered on the same chromosome, 2p12. Reg 4 is relatively uncharacterized and may have arisen by gene duplication, and is not expressed in pancreas. Reg proteins 1a and 3a are expressed only by the pancreas and are highly expressed in utero when the pancreas is being populated for the first time with islets. These specific genes are only slightly expressed during the postnatal period and are seen upregulated during times of acute pancreatic injury such as pancreatic stones and pancreatitis.

There are overlapping amino acids that exist between Reg1a and Reg3a, and their specific expression in pancreatic tissue, which indicates that these may have arisen by gene duplication, and have evolved to work together.

HIP has a signal peptide sequence which allows it to be secreted from pancreatic cells. Below in bold, is the sequence of Reg1a that is homologous to the first 6 amino acids of HIP2B. Structurally, this protein has, like Reg 3a, two “legs” and other components that may be important to its function. Without being bound by theory, it can be hypothesized that Reg 1a or Reg3a are not sufficient alone and both are required for islet neogenesis in man. The HIP sequence includes: (i) a key redundant and overlapping sequence (shown in bold below) in tandem with an exquisite and specific sequence shown (shown in italics and underlined below).

Reg 3A:
(SEQ ID NO: 26)
Mlppmalpsvswmllsclmllsqvqgeepqrelpsarircpkgskaygsh
cyalflspkswtdadlacqkrpsgnlvsvlsgaegsfvsslvksignsys
yvwiglhdptqgtepngegwewsssdvmnyfawernpstisspghcasls
rstaflrwkdyncnvrlpyvckftd.
Reg 1A
(SEQ ID NO: 27)
Maqtssyfmlisclmflsqsqgqeaqtelpqariscpegtnayrsycyyf
nedretwvdadlycqnmnsgnlvsvltqaegafvaslikesgtddfnvwi
glhdpkknrrwhwssgslvsykswgigapssvnpgycvsltsstgfqkwk
dvpcedkfsfvckfkn.
(SEQ ID NO: 28)
Human ProIslet Peptide (HIP):
iglhdptqgtepng
(SEQ ID NO: 29)
Hamster Islet Neogenesis Associated Peptide
(INGAP):
iglhdpshgtlpngs

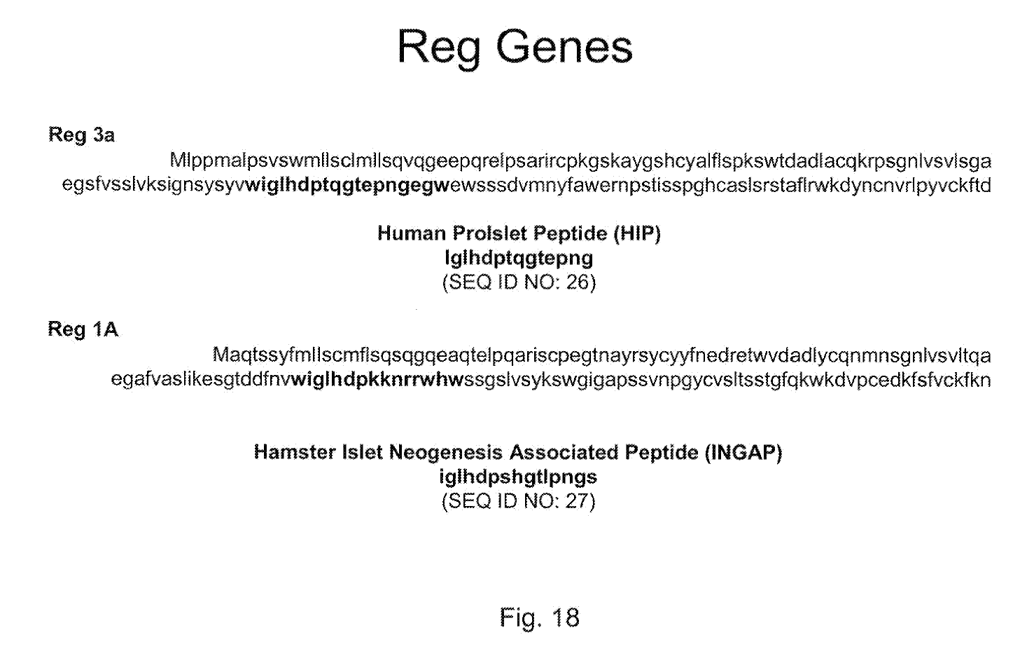

The hamster INGAP sequence was derived from expression of material from hamster pancreata and matches the reg sequences in mice and rats more closely than the reg sequences in humans (FIG. 18).

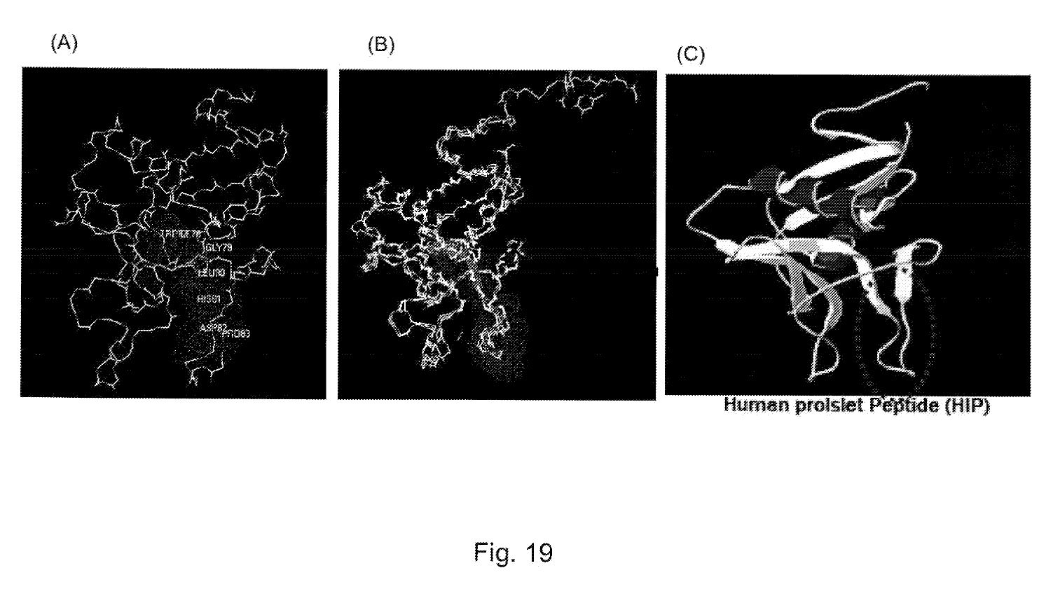

FIG. 19 represents the 3D conformations of the key human reg gene family. HIP is shown in the white dotted section on the Reg 3a structure (B). It includes the area identified in red dotted section of Reg 1a (A). The structures are remarkably similar in the predicted 3D conformation. This is the part of Reg3a that has the biological activity that was observed in preclinical investigations. Three dimensional modeling of the primary REG3a protein sequence by SwissProt folding algorithms revealed that the bioactive HIP peptide sequence is presented and exposed on the external surface of the protein (C). The HIP sequence is not folded within the confines of the protein making it available for a significant protein binding interaction and subsequent function.

The signal peptide of Reg 3a is longer than that of Reg 1a, indicating that its secretion may either delayed or its synthesis may be slower in the endoplasmic reticulum as compared to Reg 1a. HIP is shown in the red highlighted section. Reg 1a is more prevalent and more available than Reg 3a indicating that Reg 3a may be the rate limiting or pacing component to the islet regeneration pathway.

While not wishing to be bound by theory, Reg 1a alone may be sufficient to produce islet neogenesis in rodents and lower animals, but humans may require both Reg 1a in the presence of HIP, which is the bioactive portion of Reg3a, to stimulate the islet regeneration process.

The Reg3a gene is highly expressed in utero and is only ordinarily expressed postnatally in times of pregnancy and stress to the pancreas in conditions such as pancreatitis and with pancreatic stones.

The human brain accepts only glucose as its form of energy, thus there are numerous redundant mechanisms to prevent hypoglycemia which would be a lethal. Without being bound by theory, in order to turn on islet neogenesis during the postnatal period, it is hypothesized that both HIP and Reg 1a are required in the presence of specific protein mediators. Thus as a protective mechanism against hypoglycemia, islet neogenesis requires two keys to unlock the process.

Based upon the specific interaction of HIP with a generalized membrane bound protein in the presence of Reg1a and specific mediating proteins disclosed herein, it can be hypothesized without being bound by theory that only in the pancreas in the presence of HIP, can islet neogenesis proceed. The HIP sequence is bioactive and is hypothesized to be a key that interacts with both a specific membrane bound protein and Reg1a in the presence of protein mediators. Reg 1a alone does not demonstrate the ability or is not abundant enough alone without the presence of HIP to result in complete islet neogenesis. HIP acts as normally expressed Reg3a and effectively catalyzes the pathway for the islet neogenesis process to occur.

While insulin production and improved glycemic control are the end points of any compound that increases insulin production, the mechanism of action of HIP is not simply to expand or proliferate the existing beta cell population. HIP stimulates the differentiation of new islet structures that produce all four cell types contained within an islet and thus provides for not only restoration of insulin, but other modulatory hormones within the islets required for glucose homeostasis including amylin, glucagon, somatostatin and pancreatic polypeptides. Beta cells cannot live outside of a functioning islet structure, and newly formed islets are complete with a new pool of beta cells, alpha, delta and gamma cells. Once new islets are formed, beta cells within islets have the ability to proliferate.

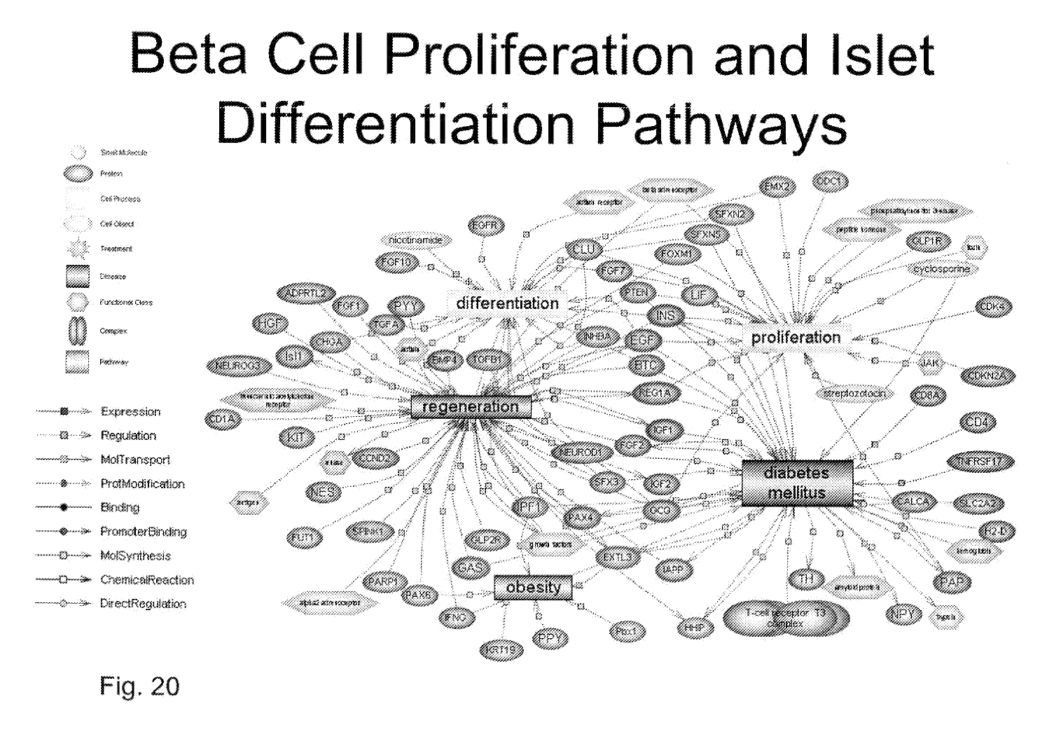

Identification of human EXTL3 as a candidate of HIP signaling was identified based on the large-scale protein-protein interaction map using in silico methodology, which evaluated databases that covered the human genes and the largest non-redundant human protein interactions. This connectivity is the culmination of unique bioinformatics processing resulting in a curated map of HIP interactors (FIG. 20-22), which has not been previously described. Other proteins involved in the islet neogenesis pathway have been identified as well as how they may be involved in the differentiation of new islets from progenitor cells. In order to identify potential receptors and protein to protein interactions with HIP, data bases of the human genome and proteome were used, which include The National Center for Biotechnology Information (NCBI) Web sites for biomedical and bioinformatics research, which hosts PubMed, GenBank, the nucleotide sequence database; Nucleotide, Protein, Gene and Map Viewer and the BLAST algorithm for sequence comparison.

Also used was the Reference Sequence database, which is a non-redundant collection of annotated DNA, RNA, and protein sequences from diverse taxa. Also the family of databases that covered the human genes was used, as well as the largest non-redundant human protein interactions which covered the human genes in a formal structure that allows searching and retrieval. This information was further curated in terms of temporal expression, and placed in spatial context with respect to cell membranes and structures.

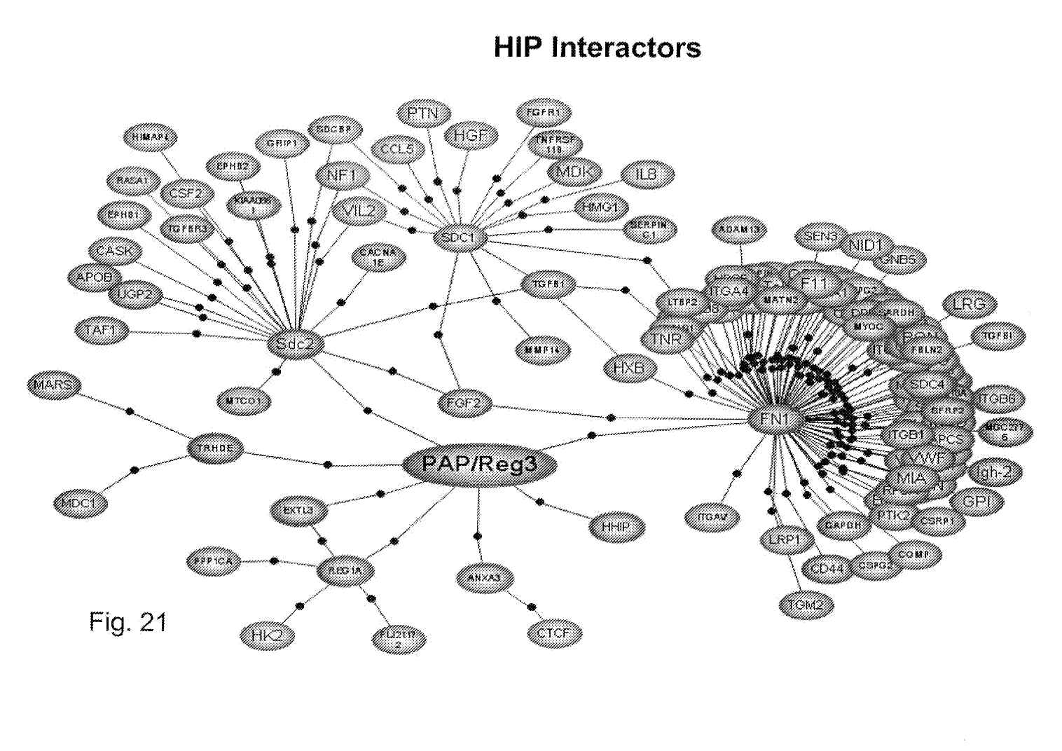

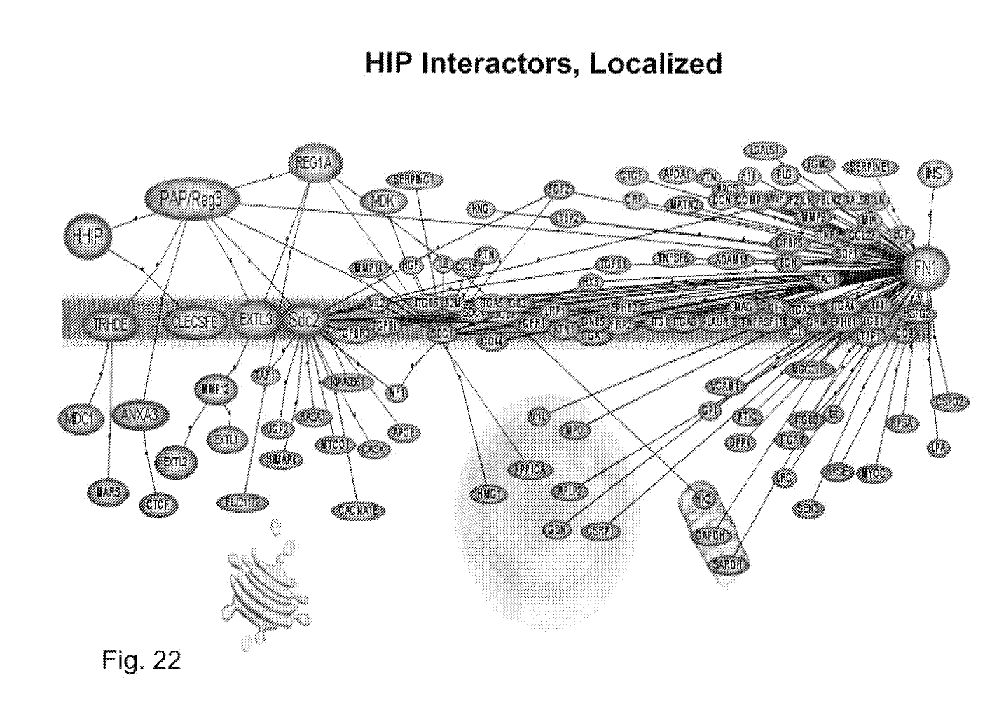

To further refine this process is to overlay data from sources that provide either, timing, action, or localization information to exclude false positive interactions that occur by methods such as yeast two hybrid screening. These methods result in minimizing false positive results before in vitro verification and confirmation of HIP interactors, which resulted in more a more refined evaluation as demonstrated in FIGS. 21 and 22. FIG. 22 demonstrates HIP interactors localized at the cell membrane with protein interactors in the intracellular and extracellular environment.

The signal pathway hypothesis that has emerged from an in silico proteomic approach, after hand refining and curation is as follows. Under conditions of a) hyperglycemia b) cytokine stimulus resulting from normal cellular apoptosis associated with the turnover of an existing islet c) under pathological conditions of pancreatic stress, pancreatitis or pancreatic stones, Reg1a and Reg1a are secreted within the pancreas and together bind and initiate an interaction with the membrane bound protein EXTL3 found on the surface of the progenitor cells of the pancreas. EXTL3 moves across the cell membrane into the intracellular matrix toward the nucleus of the cell signaling a message to differentiate into the four endocrine cell types required for islet neogenesis.

The primary candidate protein to potentially interact with HIP was identified as the human exostoses-like protein 3 (EXTL3). Consistent with this finding, EXTL3 has been implicated as a binding protein for Reg1 in rats.

It was evaluated whether HIP modulated the expression and subcellular localization of EXTL3 with a number of studies. Western blot and immunofluorescence analyses of EXTL3 localization were performed in human PANC-1 cells grown under conditions that differentiate these cells into islet-like cells. EXTL3 is predominantly located on the external surface of the cytoplasmic membrane and its structure includes a single transmembrane region located inside of the cytoplasmic membrane. PANC-1 cells were grown in a standard growth medium or in a serum free medium (SFM) containing HIP. Western blot and immunofluorescence analyses of EXTL3 localization in the PANC-1 cells revealed that when HIP was added to PANC-1 cells grown in SFM, EXTL3 appeared to translocate from the plasma membrane to the nucleus. Western blot analyses of EXTL3 levels in cytosolic and nuclear fractions were isolated. Western blot analyses demonstrated that nuclear levels of EXTL3 were observed at 6 hours after culture in SFM without HIP. Inclusion of HIP in the culture media enhanced EXTL3 nuclear translocation at 30 minutes.

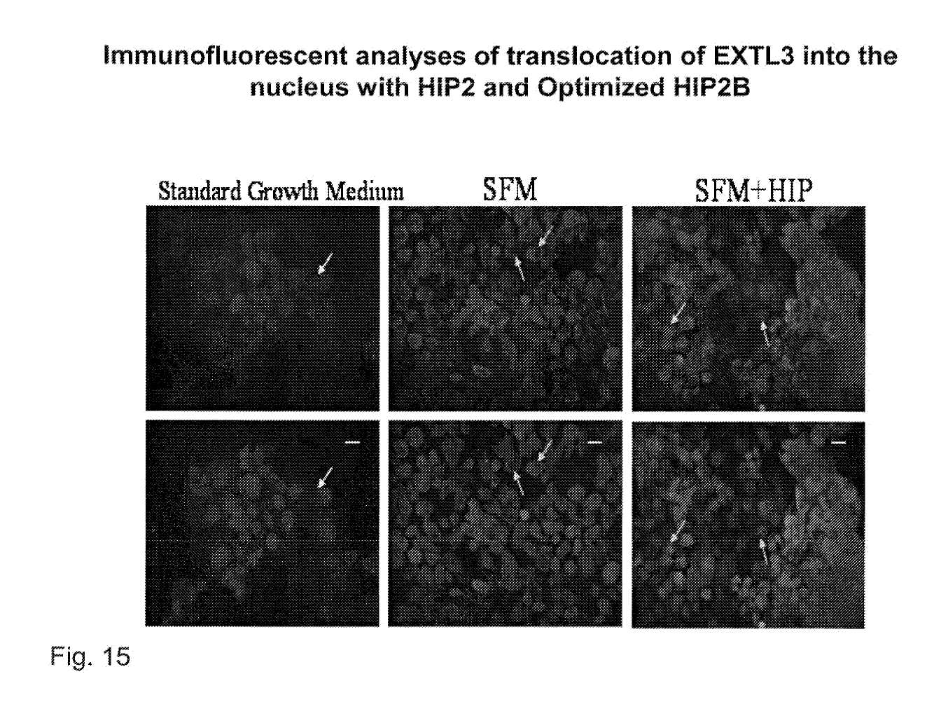

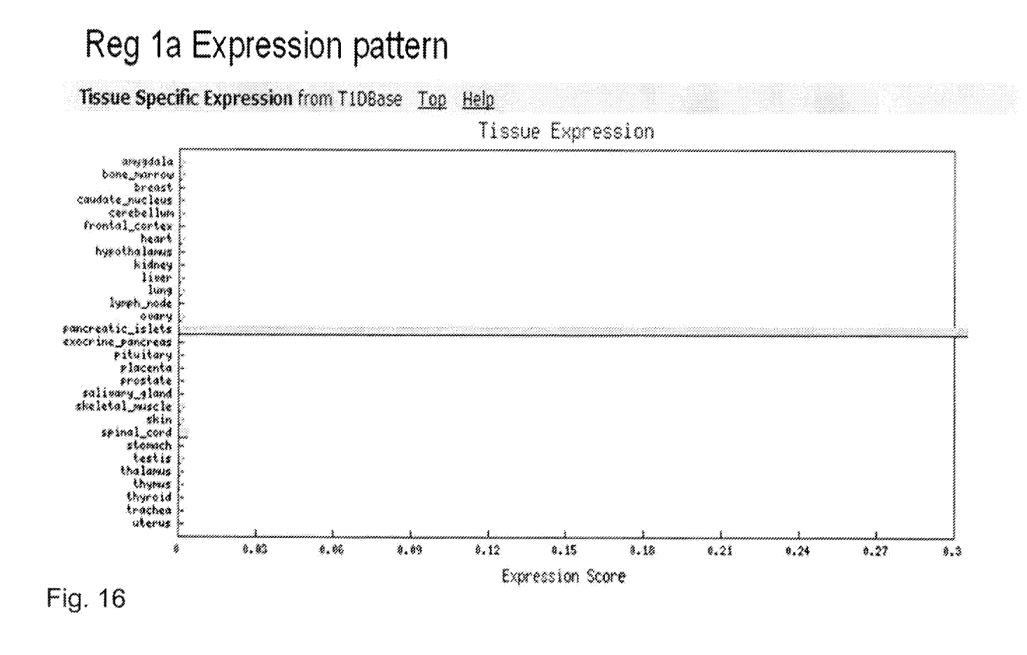

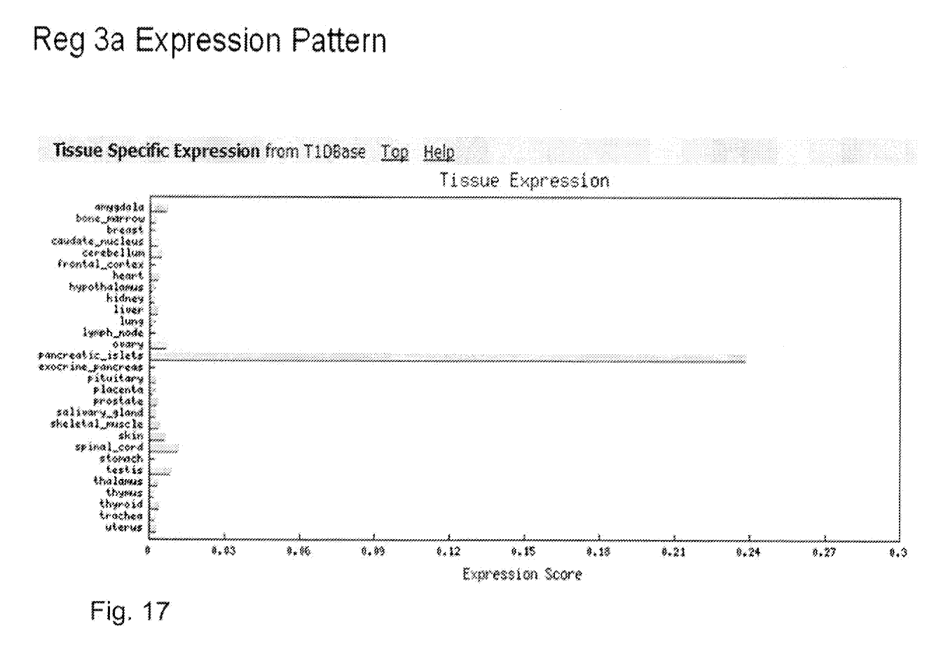

Using an antibody directed against human EXTL3, PANC-1 was immunostained and compared cells grown in SFM in the presence or absence of HIP. In standard medium, the cell borders were well-defined indicating surface expression of EXTL3 on the plasma membrane with undetectable levels of EXTL3 in the nucleus. FIG. 15 demonstrates the immunofluorescent analyses of EXTL3 are shown in the upper panels indicated by the Cy3 immunofluorescent staining of EXTL3 (red). In the lower panel of images the Cy3 immunostaining of EXTL3 has been overlaid with DAPI (blue) staining of the nuclei. Cells were grown in standard growth medium as a control and compared to cells grown serum-free medium (SFM) in the presence or absence of HIP. The yellow arrows demonstrate examples of the surface expression of EXTL3 grown in standard growth medium. The cell borders are well-defined indicating surface expression of EXTL3 on the plasma membrane. The yellow arrows delineate the cell borders while the nuclei are shown in blue. The middle images are cells grown in SFM. EXTL3 is localized in the cytoplasm as indicated by cytoplasmic Cy3 staining. The green arrows show the lack of staining in the position of the nuclei. The green arrows in the lower image of cells grown in SFM demonstrate intense blue DAPI staining of the nuclei indicating a lack of EXTL3 in the nucleus. In the upper image of the cells grown in SFM and HIP, the presence of EXTL3 immunostaining in the nucleus indicated by the blue arrows suggests a translocation of EXTL3 into the nucleus. In the lower image of cells grown in SFM and HIP, the blue arrows indicate the position of the nuclei. In the lower image there is an overlap of EXTL3-Cy3 staining and nuclear DAPI staining that corroborates the nuclear localization of EXTL3 (blue arrows). Scale bar=20 μm in all images. In cells grown in SFM and HIP, EXTL3 was present in both the cytoplasm and the nucleus as indicated by the overlap of Cy3 and DAPI staining, suggesting that HIP facilitated a translocation of EXTL3 into the nucleus.

This action also causes recruitment of proteins such as Sdc2 and FN1 involved in cell differentiation, migration, and proliferation to help form the appropriate structure necessary to form an islet. In addition, tertiary proteins, such as HHIP and ANAXA3, are recruited to promote required angiogenesis and nervous enervation required for subsequent function. After interaction with of HIP with the HIP receptor, recruitment of Reg1b, PDX-1, PAX-1, NGN3 by HIP further coordinates and regulates endocrine islet function and beta cell proliferation.

In a normally functioning pancreas, small numbers of islets die naturally on a day-by-day basis and are replaced as required to keep glucose levels under control. On average, this regenerative process replaces islets at a rate of approximately 2% per month. In nondiabetic patients, the beta cell mass within the existing islets can expand or contract depending on the insulin needs of the individual. This process is referred to as beta cell proliferation does not occur in patients with type 1 diabetes and is limited in type 2 patients. Thus the natural turnover rate is naturally too slow to allow for restoration of enough functioning beta cells without islet neogenesis.

It is important to note the distinction from beta cell regeneration/proliferation, which occurs within the beta cell compartment of existing islets; the interaction between HIP with the HIP receptor protein results in islet neogenesis, which is the formation of new four-celled islet structures, the beta cells are but one of the four cell compartments. Each of the cell types within the islet secrete hormones, which are involved in metabolism and include insulin, amylin, glucagon, somatostatin and pancreatic polypeptides. Beta cell regeneration and expansion of beta cells within existing islets may improve insulin production, whereas islet neogenesis restores all four cell types, their hormones and metabolic perterbations resulting from diminished islet mass and function.

Islet neogenesis occurs as a result of differentiation of progenitor cells that exist within the adult pancreas. While insulin production and improved glycemic control are the end points of any compound that increases insulin production, the mechanism of action of HIP is not simply to expand or proliferate the existing beta cell population. HIP stimulates the differentiation of new islet structures that produce all four cell types contained within an islet and thus provides for not only restoration of insulin, but other modulatory hormones within the islets required for glucose homeostasis including amylin, glucagon, somatostatin and pancreatic polypeptides. Beta cells cannot live outside of a functioning islet structure, and a newly formed islets are complete with a new pool of beta cells, alpha, delta and gamma cells. Once new islets are formed, beta cells within islets have the ability to proliferate.

The effect of HIP is highly leveraged by the power of acting upon progenitor cells, which exist within the adult human pancreas. The presence of these progenitor cells throughout the adult pancreas capable of differentiating into new islets has been demonstrated.

The existence of a gene in rat regenerating islets that is highly specific to the normal replication of pancreatic cells but not related to malignant growth of pancreatic cells nor regeneration of other tissues has been identified. There has been a similar human gene found in man to the rat gene, which is referred to as the Reg gene, and is the gene upon which HIP has been found in humans and a high homology between species of Human proIslet Peptide and other mammalian species. The following species homology chart shows that HIP is a sequence that is exquisitely conserved through evolution. It is likely that each of these sequences will have some efficacy in humans, but none is exactly matched to the human active sequence of HIP.Species Homology

| | | |
|---|---|---|
| Human | W I G L H D P T Q G T E P N G E | SEQ ID NO: 30 |
| Chimp | W I G L H D P T Q G S E P D G G | SEQ ID NO: 31 |
| Hamster | W I G L H D P S H G T L P N G S | SEQ ID NO: 32 |
| Mouse | W I G L H D P T M G Q Q P N G G | SEQ ID NO: 33 |
| Norway | W I W L H D P T M G Q Q P N G G | SEQ ID NO: 34 |
| Rat | | |
| Cow | W I G L H D P T E G S E P D A G | SEQ ID NO: 35 |
| Dog | W N G L H D P T E G Y E P N A D | SEQ ID NO: 36 |
| Sheep | W I G L H D P T E G S E P N A G | SEQ ID NO: 37 |

HIP is highly conserved among mammals. Of the amino acid substitutions between the human and other species, each amino acid that varies is the result of a single nucleotide polymorphism (SNP). In most cases, the surviving SNP results in an amino acid that is either in the same class or closely related. For example, the methionine (M) is another non-polar hydrophobic amino acid like isoleucine in the second position of the dog sequence. Serines and threonines are sometimes swapped, but these are both polar uncharged amino acids. The switch of asparginine and aspartic acid is a SNP, as are all other amino acid substitutions across all species.

In one embodiment, the HIP peptides are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7 membered alkyl, amide, amide lower alkyl, amide di (lower alkyl), lower alkoxy, hydroxy, carboxy and the lower ester derivatives thereof, and with 4-, 5-, 6-, to 7 membered heterocyclics. For example, proline analogs can be made in which the ring size of the proline residue is changed from 5 members to 4, 6, or 7 members. Cyclic groups can be saturated or unsaturated, and if unsaturated, can be aromatic or nonaromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24,243-252,1989). Certain peptidomimetic compounds are based upon the amino acid sequence of the peptides disclosed herein. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity, i.e., binding to the HIP receptor, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Peptidomimetic design strategies are readily available in the art (see, e.g., Ripka & Rich, Curr. Op. Chem. Biol. 2,441-452,1998; Hruby et al., Curr. Op. Chem. Biol. 1,114-119,1997; Hruby & Balse, Curr. Med. Chem. 9,945-970,2000). One class of peptidomimetics a backbone that is partially or completely non-peptide, but mimics the peptide backbone atom-for atom and comprises side groups that likewise mimic the functionality of the side groups of the native amino acid residues. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich,1998, supra).

In one embodiment, HIP peptides, including, but not limited to HIP1, HIP2 and HIP3, or peptidomimetics thereof, can be used for both diagnosis and therapy. Non-limiting examples of the effects of such HIP peptides include islet neogenesis, treatment of type 1 or type 2 diabetes, and other pathologies related to aberrant glucose, carbohydrate and/or lipid metabolism, impairment in insulin secretion or action, including insulin resistance at the level of the adipose tissue, muscles or liver, including fasting hyperglycemia, insulin resistant syndrome, hyperglycemic conditions generally in children or adults and those with a family history of diabetes exhibiting an abnormal fasting glucose or insulin levels, metabolic syndrome, being overweight, obesity, polycystic ovarian syndrome (PCOS), anovulatory cycles, fasting hyperlipidemia/hypercholesterolemia, elevated fasting total cholesterol, elevated LDL and VLDL cholesterol, family history of diabetes and some forms of impotence and sexual dysfunction associated with such conditions.

Another embodiment of the present disclosure provides peptidomimetics of mediating proteins, including, but not limited to, Reg1a, syndecan 2, fibronectin 1, annexin A3, and Hedgehog-interacting protein. Such peptidomimetics may be useful for diagnosis and therapy.

Further embodiments of the present disclosure relate to methods of stimulating islet neogenesis, and treat type 1 and type 2 diabetes and other pathologies associated with aberrant glucose metabolism comprising administering mediating proteins, including, but not limited to, Reg1a, syndecan 2, fibronectin 1, annexin A3, and Hedgehog-interacting protein, or peptidomimetics thereof.

Embodiments of the present disclosure also feature a method and assay for rational drug design of agents that can, like the HIP peptides, bind to the HIP receptor, thereby stimulating islet neogenesis and treat type 1 and type 2 diabetes and other pathologies associated with aberrant glucose metabolism.

Thus, according to another embodiment of the disclosure, a method is provided for making a drug suitable for treating type 1 and type 2 diabetes and other pathologies associated with aberrant glucose metabolism in a mammal comprising (a) constructing a compound that binds a mammalian HIP receptor and stimulates islet neogenesis and (b) determining whether the compound stimulates islet neogenesis, an affirmative determination indicating that the drug is suitable for treating the diseases.

The disclosure also provides a method of screening for a compound that binds a HIP receptor. The method comprises: (a) providing a synthetic HIP peptide or other cognate ligand and a HIP receptor to which it binds; (b) combining the peptide/ligand and the HIP receptor in the presence of a test compound under conditions wherein, in the absence of the test compound, a pre-determined quantity of the HIP peptide would bind the HIP receptor; and (c) determining if the quantity of the HIP peptide bound to the HIP receptor is decreased in the presence of the test compound, the decrease being indicative that the test compound binds the HIP receptor and stimulate islet neogenesis. This method may comprise additional assay steps, such as determining if the test compound modulates islet neogenesis or is capable of modulating glucose-related diseases.

A further embodiment of the present disclosure also feature a method and assay for rational drug design of agents that can, mediate binding of the HIP peptides to the HIP receptor, thereby stimulating islet neogenesis and treat type 1 and type 2 diabetes and other pathologies associated with aberrant glucose metabolism. Such agents, also referred to as mediating proteins, include, but not limited to, Reg1a, syndecan 2, fibronectin 1, annexin A3, and Hedgehog-interacting protein, or peptidomimetics thereof.

Another embodiment of the present disclosure provides a method of screening for a compound that mediates binding of HIP to the HIP receptor.

### EXAMPLE 1

Methods: In order to identify potential receptors and protein to protein interactions with HIP, both proprietary and publicly available data bases of the human genome and proteome were searched including The National Center for Biotechnology Information (NCBI) Web sites for biomedical and bioinformatics research, which hosts PubMed, GenBank, the nucleotide sequence database; Nucleotide, Protein, Gene and Map Viewer and the BLAST algorithm for sequence comparison. Also utilized was The Reference Sequence database, which is a non-redundant collection of annotated DNA, RNA, and protein sequences from diverse taxa. Also evaluated was a family of databases that covered the human genes and the largest non-redundant human protein interactions which covered the human genes in a formal structure that allows searching and retrieval.

It was determined that the putative HIP pathway and protein interactors by evaluating and hand curating all potential interactors with HIP and the Reg3a gene in humans. By the integration of molecular mechanisms, gene and protein expression analysis and integration of multiple heterogeneous data sets, all of the potential protein interactors, as well as proteins in the vicinity of and have direct or indirect interactions with HIP and the HIP receptor, were identified. Additional, in vitro studies were conducted with differential pulldown analyses along the time points in cell response to HIP stimuli.

Amino Acids 1-919 of the HIP receptor are listed below:

(SEQ ID NO. 38)
MTGYTMLRNGGAGNGGQTCMLRWSNRIRLTWLSFTLFVILVFFPLIAHYY
LTTLDEADEAGKRIFGPRVGNELCEVKHVLDLCRIRESVSEELLQLEAKR
QELNSEIAKLNLKIEACKKSIENAKQDLLQLKNVISQTEHSYKELMAQNQ
PKLSLPIRLLPEKDDAGLPPPKATRGCRLHNCFDYSRCPLTSGFPVYVYD
SDQFVFGSYLDPLVKQAFQATARANVYVTENADIACLYVILVGEMQEPVV
LRPAELEKQLYSLPHWRTDGHNHVIINLSRKSDTQNLLYNVSTGRAMVAQ
STFYTVQYRPGFDLVVSPLVHAMSEPNFMEIPPQVPVKRKYLFTFQGEKI
ESLRSSLQEARSFEEEMEGDPPADYDDRIIATLKAVQDSKLDQVLVEFTC
KNQPKPSLPTEWALCGEREDRLELLKLSTFALIITPGDPRLVISSGCATR
LFEALEVGAVPVVLGEQVQLPYQDMLQWNEAALVVPKPRVTEVHFLLRSL
SDSDLLAMRRQGRFLWETYFSTADSIFNTVLAMIRTRIQIPAAPIREEAA
AEIPHRSGKAAGTDPNMADNGDLDLGPVETEPPYASPRYLRNFTLTVTDF
YRSWNCAPGPFHLFPHTPFDPVLPSEAKFLGSGTGFRPIGGGAGGSGKEF
QAALGGNVPREQFTVVMLTYEREEVLMNSLERLNGLPYLNKVVVVWNSPK
LPSEDLLWPDIGVPIMVVRTEKNSLNNRFLPWNEIETEAILSIDDDAHLR
HDEIMFGFRVWREARDRIVGFPGRYHAWDIPHQSWLYNSNYSCELSMVLT
GAAFFHKYYAYLYSYVMPQAIRDMVDEYINCEDIAMNFLVSHITRKPPIK
VTSRWTFRCPGCPQALSHDDSHFHERHKCINFFVKVYGYMPLLYTQFRVD
SVLFKTRLPHDKTKCFKFI.

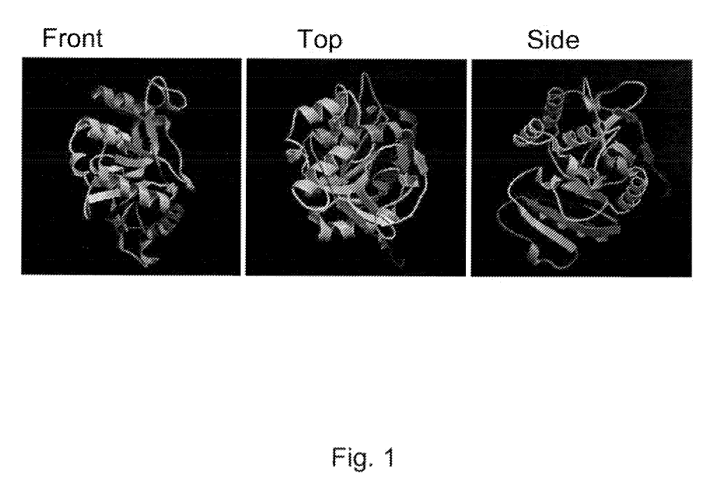

The 3D structure of the human EXTL3 gene is shown in FIG. 1.

### EXAMPLE 2

Leading to the demonstration of HIP binding to the HIP receptor with a cascade of signals from the cell membrane to the nucleus leading to islet neogenesis, demonstration of efficacy of HIP both in vitro and in vivo was necessary. Based upon the methods presented, it has been identified that HIP interacts with the EXTL3 protein located within the cell membrane of human PANC-1 cells. Utilizing a technique of growing an established immortalized human cell line from human pancreatic epithelioid cells known as PANC-1, the impact in vitro of impact of HIPs and Optimized HIPs on insulin production was evaluated. This cell line demonstrates the ability to differentiate into other pancreatic cell types upon appropriate signaling. Therefore, PANC-1 cells were used as a surrogate to the naturally occurring progenitor cells of the pancreas.

In Vitro Proof of Concept of HIP Activity. Human pancreatic islet and ductal fractions were cultured over 10 days and then treated in a blinded study. Radioimmunoassay methods were used to measure insulin levels in the human pancreatic cultures treated with a scrambled peptide serving as a negative control, HIP3, HIP1, HIP2 and hamster-derived INGAP serving as a positive control. Peptides were synthesized (95% pure, research grade).

Duplicate cultures were treated in both ductal and islet fractions of human pancreatic tissue on day 10 and day 12 and then lysed for detection of insulin content after 1 week of treatment HIP peptides, control and INGAP. During 10-day culture, the insulin production goes down and then after treatment with HIP peptides, insulin is produced again.

The ductal and islet tissue were separated using the Ricordi method. Neither ductal cell nor islet culture was completely homogeneous in nature. The studies also suggest that progenitor cells, which are the target for HIP, are found both in islet and ductal cultures. The studies were repeated with similar findings shown in the following chart, with as much as a four-fold increase in insulin levels by radioimmunoassay among human ductal tissue cultured with HIP2.

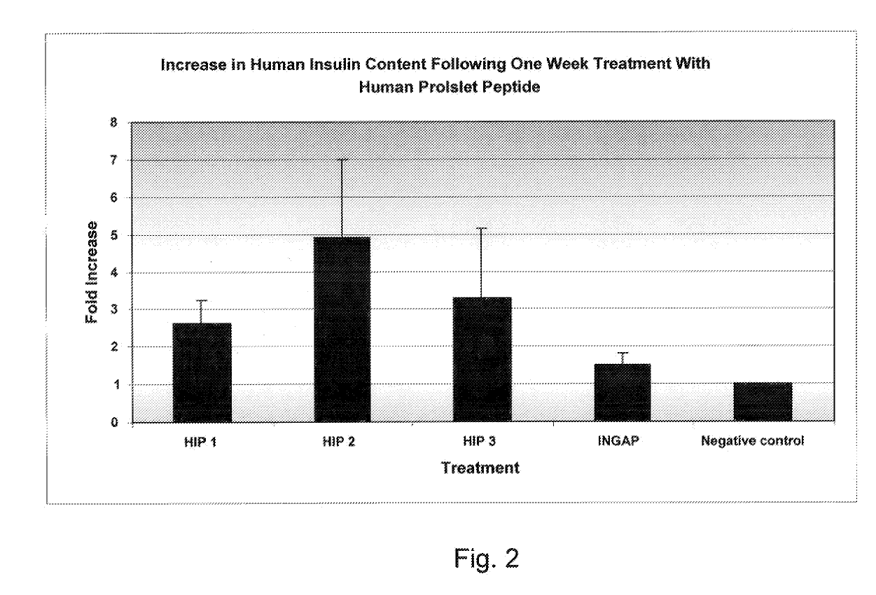

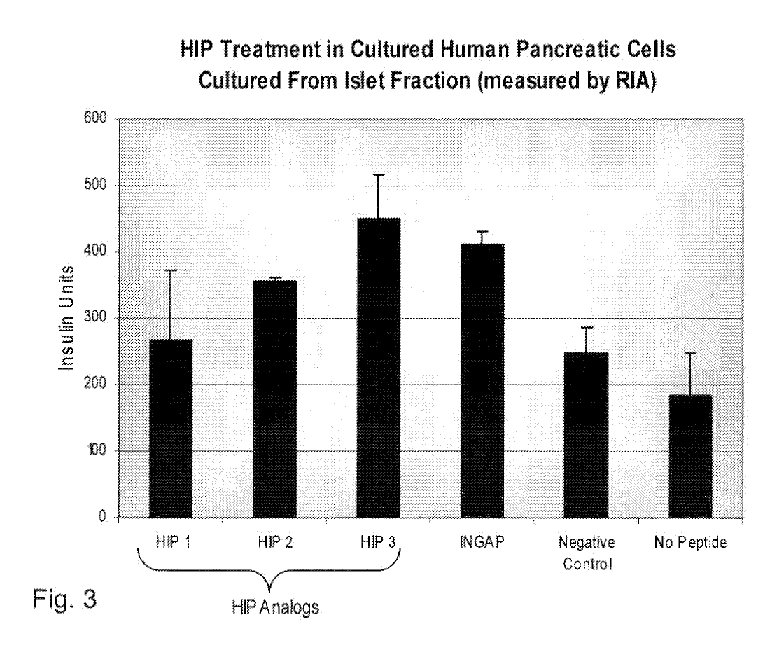

The ductal fraction graph as shown in FIG. 2 depicts the insulin levels on the y axis as measured by radioimmunoassay after incubation in culture with human pancreatic ductal tissue. The islet fraction graph indicates insulin levels after incubation in human pancreatic islet tissue. Baseline insulin levels are significantly higher in the islet fraction at baseline than in the ductal fractions at baseline. Similar studies were conducted with HIP and hamster INGAP in islet fractions, as shown in FIG. 3.

Repeated studies confirmed the increase in insulin both in predominately human ductal cell cultures and islet cultures, with baseline insulin levels consistently about ⅓ lower in the baseline ductal cultures compared to islet cultures, with similar rises in insulin content after incubation with HIP peptides compared to a negative control.

### EXAMPLE 3

In Vivo Studies. HIP3, HIP1, HIP2 and hamster INGAP have been the subject of in vivo studies in mice. Studies have shown that these HIP variants, when introduced into diabetic mice, stimulate differentiation of progenitor cells within the pancreas into new islet structures A model of diabetes has been developed in the mouse. The subject number was selected to yield a sufficient number of diabetic animals for the study and animals were randomly assigned to study groups. All animals were dosed via intraperitoneal injections twice daily (am and pm) for 28 consecutive days. The timing of dose administration remained consistent (±2 hours) during the dosing phase. After confirmation that the mice had been diabetic (blood glucose greater than 16.7 mmol/L (300 mg/dL) for at least 1 week, mice were dosed.

Mice were injected intraperitoneally with streptozocin at 40 mg/kg in citrate buffer, pH 4.5, on 5 consecutive days in an attempt to render them diabetic. Mice must have had blood glucose greater than 16.7 mmol/L (300 mg/dL) for at least 1 week to be considered diabetic. If the blood glucose level in any animal rose to above 400 mg/dL, the animal was treated with insulin. Every 3 days, at the same time each day, a nick was made on the tail and a drop of blood was collected. Glucose measurements were determined using a glucose meter. Group assignments and dose levels were as follows in Table 1:

TABLE 1

| | | Dose | | Dose | Number of |
|---|---|---|---|---|---|
| Group | Treatment | Level | | Volume | Animals |
| 1 | Vehicle | 0 | | 100 μl | 6 |
| 2 | HIP3 | 250 | μg | 100 μl | 6 |
| 3 | HIP1 | 250 | μg | 100 μl | 6 |
| 4 | HIP2 | 250 | μg | 100 μl | 6 |
| 5 | Hamster | 250 | μg | 100 μl | 6 |
| | INGAP | | | | |

Study endpoints included the following: changes in glucose; changes in insulin requirements; and histology of post-mortem pancreata.

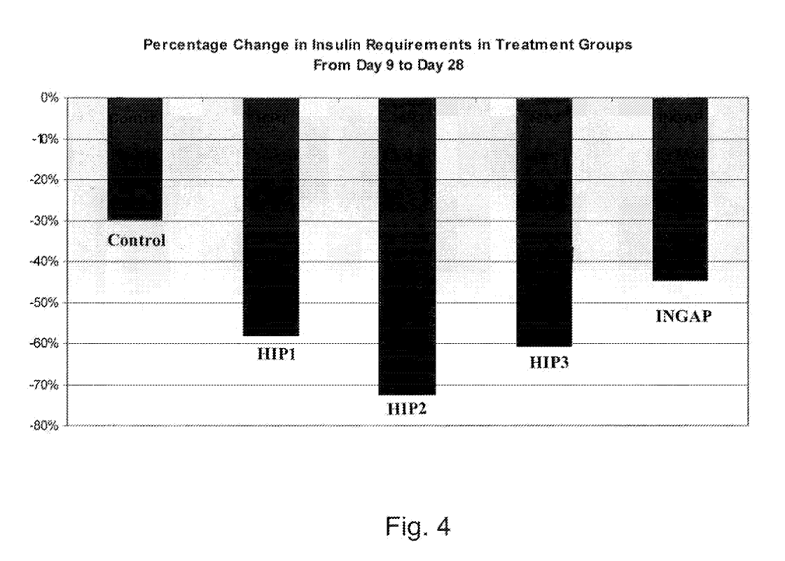

Changes in Insulin Requirements. Reductions in both the insulin requirements and the rate of decrease in insulin requirements were seen among HIP-treated mice and hamster INGAP and placebo-treated groups. FIG. 4 demonstrates the reduction in insulin dosages with HIP2-treated mice being completely insulin-free by day 21.

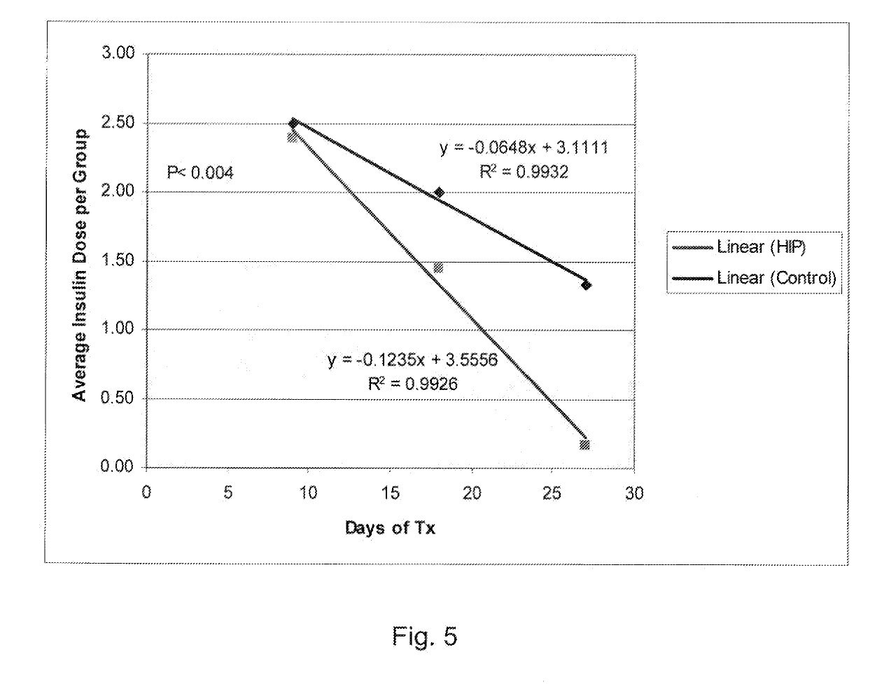

FIG. 5 demonstrates the rate of reduction of insulin required by mice was also significantly faster in HIP-treated diabetic mice compared to control (p=0.004). Based upon glucose levels in mice, insulin was administered, and there were concomitant reductions changes in glucose levels, which are reflected in the reduced need for insulin. There was a 14.7% lower mean glucose between HIP1 and control, a 29.4% lower mean glucose between HIP2 and control, and a 57.3% mean lower glucose between HIP3 and the control group. The data indicates the significantly faster rate of decline in insulin requirements among all HIP-treated mouse groups compared to control diabetic mice. There were significantly greater numbers of islets after HIP treatment observed in mouse pancreata, which were sectioned and reviewed on each mouse studied. The pancreata were evaluated by a histologist blinded the specimens with the following data shown in Table 2. Initial Study of the impact of HIP on islet mass and number.

TABLE 2

| | | Total Islet Mass |
|---|---|---|
| | Total Islets (% | (μm2) (% |
| | increase) | increase) |
| Placebo | 280 | 854364 |
| HIP2 | 454 (62%) | 2161782 (153%) |
| HIP3 | 410 (46%) | 1703513 (99%) |

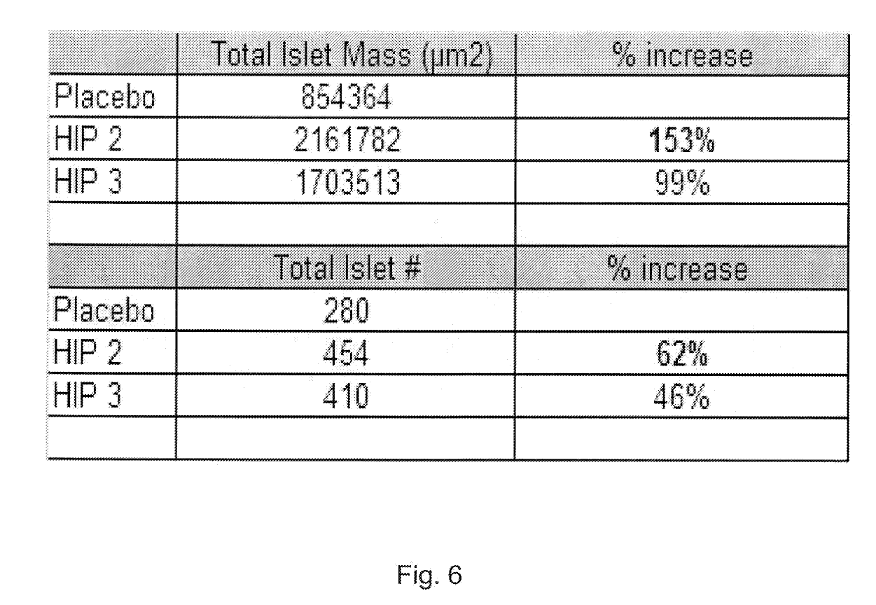

The difference in islet number between HIP and placebo was statistically significant (p=0.022) (Table 2). There was even a more profound increase in islet area between the HIP-treated mice and the placebo-treated group. The islet area in the HIP2-treated group was 360,297 μm(2) compared to 142,394 μm(2) in the placebo-treated group with 283,918 μm(2) in the HIP3-treated group (p=0.05), as shown in FIG. 6.

Repeated study among streptozotcin-treated mice rendered diabetic with HIP2B (Optimized HIP) demonstrated a three-fold rise in the total number of islets compared to placebo-treated mice (Table 3). Differences between the two groups of HIP2B and placebo were statistically significant for both an increase in total islet mass (p=0.040) and total islet numbers (p=0.032), with the HIP2B group indicating larger values for both islet mass (M=416714.67, SD=121,389.01) and islet number (M=94, SD=32.74) than for the placebo group (islet mass: M=127410.67, SD=96930.78, islet number: M=31.67, SD=15.28).

The islet sizes between the HIP2B/Optimized HIP treated diabetic mice and placebo-treated mice, were not statistically significant on the dependent variable of islet size (t=0.708, p=0.518) indicating that normally sized islets were generated by HIP2B/Optimized HIP. Mann-Whitney U tests confirmed the finding of the t-tests.

TABLE 3
Study evaluating the Impact of HIP2 and Optimized HIP2B on
islet Mass and number

| | | Total Islet Mass |
|---|---|---|
| | Total Islets (% | (μm2) (% |
| | increase) | increase) |
| Placebo | 35 | 83,643 |
| HIP2 | 65 (86%) | 202,955 (143%) |
| HIP2B | 94 (168%) | 416,715 (398%) |

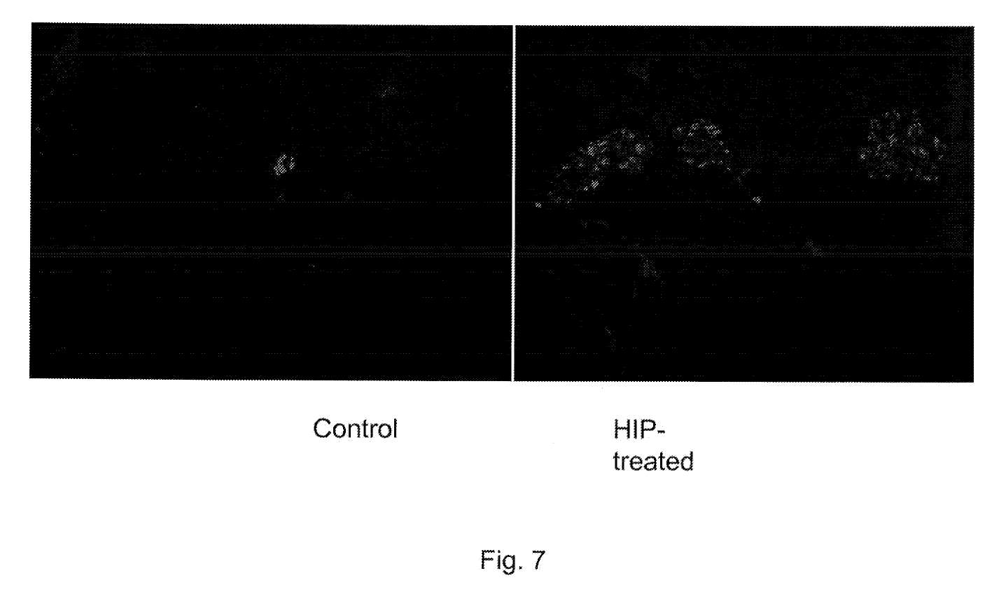

Immunofluorescent staining for insulin was also performed on mouse pancreata demonstrate are greater degree of insulin staining in the HIP-treated mice, as shown in FIG. 7. This mouse pancreas tissue was harvested and fixed in 4% PFA, blocked and sectioned. 10X-Objective, 1.6 optivar.

### EXAMPLE 4

Utilizing a technique of growing an established immortalized human cell line from human pancreatic epithelioid cells known as PANC-1, the impact in vitro of impact of HIPs and Optimized HIPs on insulin production was evaluated. This cell line demonstrates the ability to differentiate into other pancreatic cell types upon appropriate signaling. Therefore, PANC-1 cells were used as a surrogate to the naturally occurring progenitor cells of the pancreas.

PANC-1 cells were seeded in T75 flasks in DMEM media containing 10% fetal bovine serum. The cells were incubated at 37° C., 5% CO2 for 24 hours and then treated with HIPs at the final concentration of 167 nM. This treatment was performed once a day for four days. On the fifth day the cells were broken to obtain the cell lysates. In these cell extracts the total protein levels were determined, and 50 micrograms of total protein were used to perform the western blot analysis. The samples containing 50 micrograms of proteins were diluted in loading buffer containing or not 5% of the reducing agent beta-mercaptoethanol, and loaded into each well of the gel. After the electrophoresis and transfer of the proteins to nitrocellulose membranes, the presence of insulin was detected by using as a primary antibody the polyclonal chicken anti insulin antibody (ab14042, dilution of 1/2000), and as secondary antibody the rabbit polyclonal-HRP conjugated anti-chicken (dilution 1/1000 for the NIT gel and 1/2000 for the PANC-1 gel).

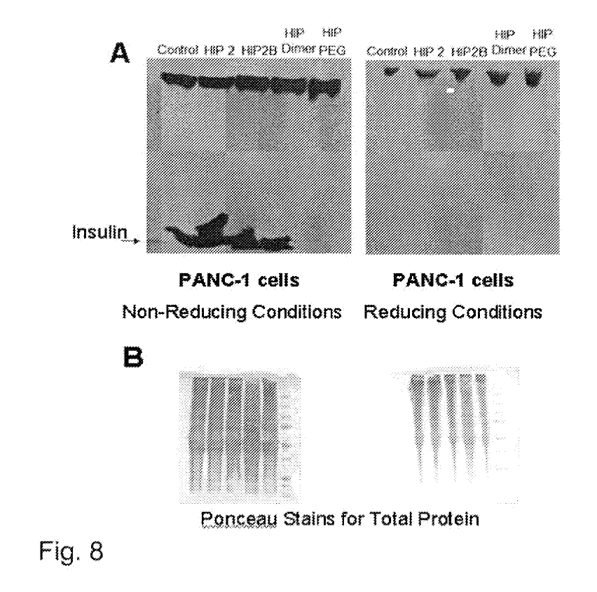

FIG. 8A is a Western Blot analysis demonstrating expression of human insulin from PANC-1 cells in response to incubation with HIP and Optimized HIP under non-reducing and reducing conditions. The panel labeled A demonstrates bands for insulin in the PANC-1 cells when the samples were loaded in non-reducing conditions. FIG. 8B are Ponceau Stains under non-reducing and reducing conditions in response to incubation with various HIP and Optimized HIPs.

FIG. 8B show the total protein contained in the same membranes as in FIG. 8A. The determination of the levels of total protein via Ponceau staining demonstrates that the different lanes contain similar amount of proteins. The total protein levels in the NIT-1 and PANC-1 cells were determined, and 50 micrograms of total protein were used to perform the Western Blot analysis. The samples containing 50 micrograms of proteins were diluted in loading buffer containing or not 5% of the reducing agent beta-mercaptoethanol, and loaded into each well of the gel.

The Ponceau staining demonstrates that the differences in insulin expression respond to different HIPs and Optimized HIPs are not related with amount of protein loaded in the wells. Also the lack of signal for insulin, for example, the membrane in the reducing conditions, is not attributed to a lack of protein.

### EXAMPLE 5

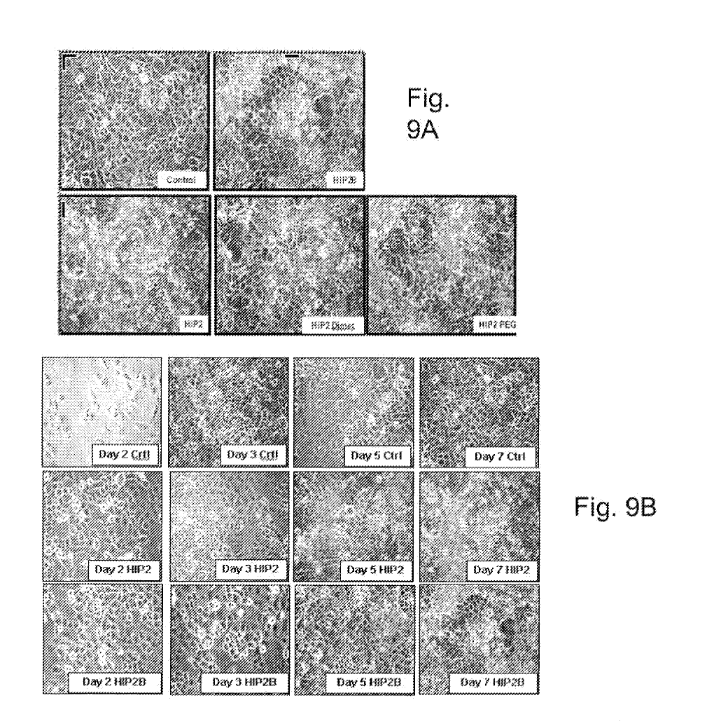

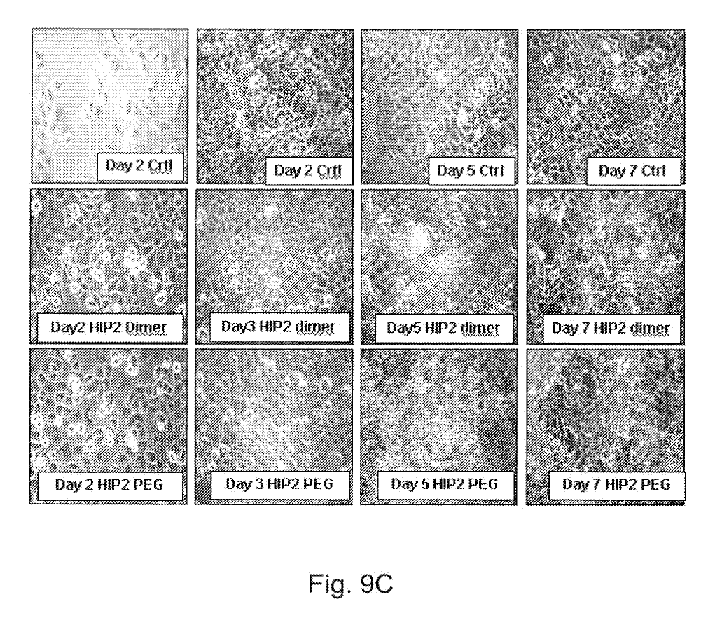

FIG. 9 demonstrates the impact of HIP and Optimized HIP Peptides on Cell Morphology in human PANC-1 Cell Lines. The cells were treated with HIP and Optimized HIP peptides for four days. In FIG. 9A, taken on day 7 at 200× magnification, morphological differences can be seen between the control condition and the cells treated with HIPs and Optimized HIPS with histologically more differentiated cells, particularly in the HIP2B-treated cells. FIG. 9B show the progression of the cell morphology changes through 7 days, with the control on the top, HIP2 in the middle, and HIP2B on the bottom. Pictures were taken on days 1, 2, 3, 5, and 7 at 200× magnification. While the control-treated cells did not appear to undergo any changes, the cells treated with HIP2 and HIP2B deviate significantly from their initial appearance. FIG. 9C demonstrates the progression of morphological changes when HIP2 Dimer and HIP2 PEG are treated in PANC-1 cell cultures. Overall, the control-treated cells did not undergo any significant visual changes, the cells treated with HIP2 and HIP2B deviate significantly from their initial appearance.

### EXAMPLE 6

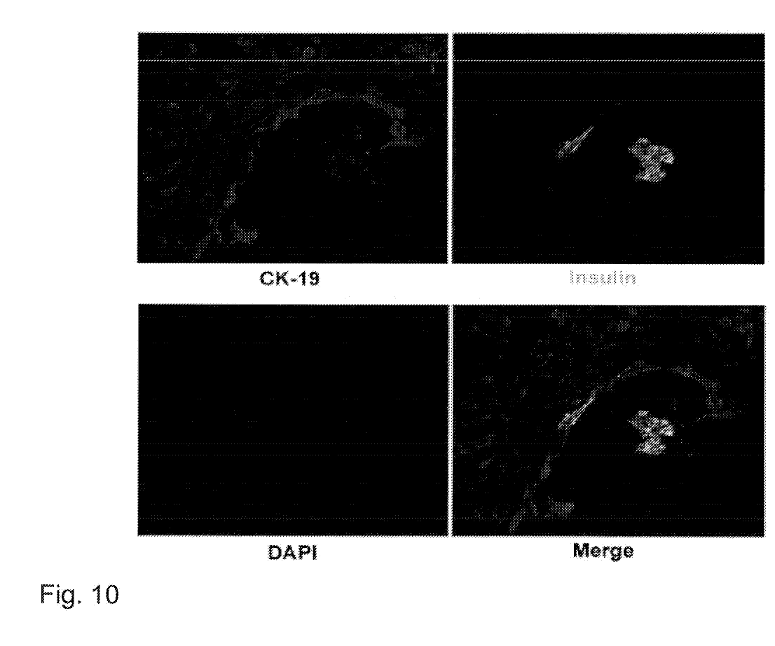

FIG. 10 demonstrates HIP2B Activity in Human Pancreatic Tissue Culture. Studies demonstrated the impact of HIP and Optimized HIP peptides in, human pancreatic ductal cell cultures. The ductal fraction of human pancreatic cells were cultured for 10 days in a collagen matrix and then treated every other day with HIP2B. Cells were labeled by double antibody staining for CK 19, a marker for ductal tissue, and DAP1 staining to show nuclei and insulin. As shown in FIG. 10, the cells underwent morphological changes that induced insulin expression in otherwise insulin negative cells.

### EXAMPLE 7

Pilot Data of Impact of HIP and Optimized Hip Peptides in the Non Obese Diabetic Model.

Consistent with the data in the STZ-treated mice (above) of increased islet mass, area and number, the pilot NOD mouse model demonstrated preliminary evidence of the potential for Optimized HIP to provide better efficacy in terms of islet neogenesis as measured by C-peptide levels in mice following HIP treatment for 39 days.

The non obese diabetic (NOD) model is used as a model for type 1 autoimmune diabetes. This form of diabetes is the most challenging in that the underlying damage to the pancreas and its insulin production is due to autoimmune attack. Therefore, in order to show definitive islet neogenesis in this form of diabetes an immune tolerance agent must be used in combination with HIP. The NOD mouse model is extremely difficult model because many of the mice may only transiently become diabetic and go into remission, whereas others develop severe diabetes. The timing intervention in this transgenic mouse model is difficult to determine.

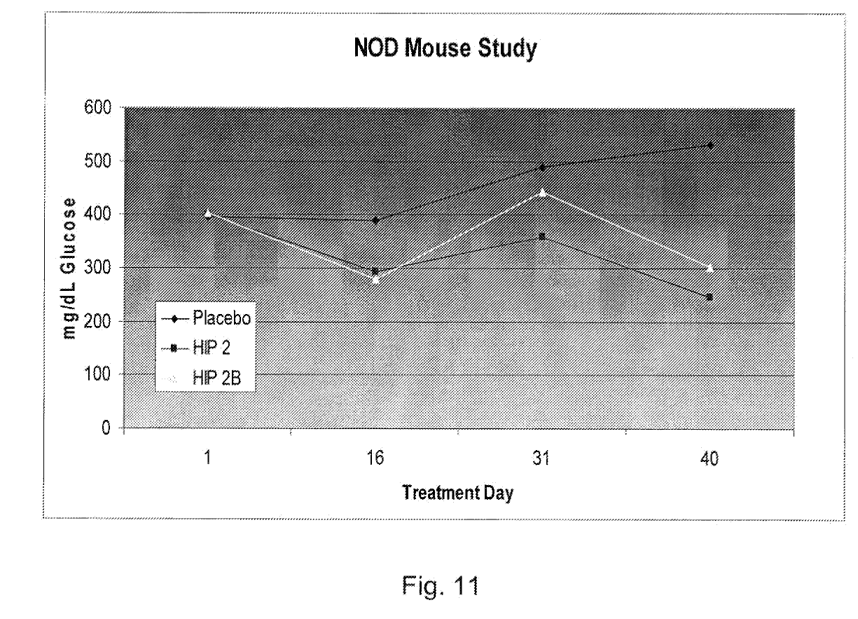

In a preliminary study that utilizing the immune tolerance agent, lysophylline (LSF) under development, three NOD mice that became diabetic and were randomized to placebo plus LSF, HIP2 plus LSF and HIP2B plus LSF. As shown in FIG. 11, of the group who received LSF at the appropriate time, the two treated with HIP responded with steadily improved glucose levels during the study compared to the NOD mouse treated with LSF alone that had gradual elevations in glucose throughout the study. While not a statistically significant study, these data provide very compelling evidence for pursuit of the combination of an immune tolerance agent and HIP for type 1 diabetes.

### EXAMPLE 8

Impact of HIP2B and HIP2 on HIP Receptor. To provide evidence that EXTL3 interacts with HIP, an experiment was conducted where human ductal cell line PANG-1 was used as a source of EXTL3. Using an antibody directed against EXTL3, PANC-1 cells were immunostained after being grown under conditions that differentiate these cells into islet-like cells. PANC-1 cells were grown in a standard growth medium containing 10% fetal bovine serum, or in a serum free medium (SFM) or SFM containing HIP. It was observed that EXTL3 is expressed in PANC-1 cells and that this expression increases when the cells are grown in SFM Immunofluorescence analysis of EXTL3 localization in the PANC-1 cells revealed that EXTL3 appeared to translocate from the plasma membrane to the nucleus in response to HIP.

The following sets of studies demonstrate that HIP2B is as effective as HIP2 in the interaction with the cytoplasmic membrane receptor/EXTL3 for HIP and trafficking from the receptor to the nucleus. The receptor for Human ProIslet Peptide was labeled using a double antibody method in a stable human pancreatic cell line. The first antibody was a rabbit polyclonal and the second was a goat-anti-rabbit labeled with Cy3 fluorescent dye.

These cells grow normally in serum free media and when treated with trypsin, are destabilized and made competent to undergo developmental changes. Cells were cultured in serum free media (SFM) with and without HIP, and in serum free media with trypsin (TSFM). This is to show that simply destabilizing, does not activate developmental changes.

When treated with HIP under stable conditions, no changes result. When treated with HIP under developmentally competent conditions, the labeled receptor responds to the presence of HIP by being encapsulated by the cytoplasmic membrane and moving to the nuclear membrane where the signals for differentiation are received.

When treated with HIP under stable conditions, no changes result. When treated with HIP under developmentally competent conditions, the labeled receptor responds to the presence of HIP by being encapsulated by the cytoplasmic membrane and moving to the nuclear membrane where the signals for differentiation are received.

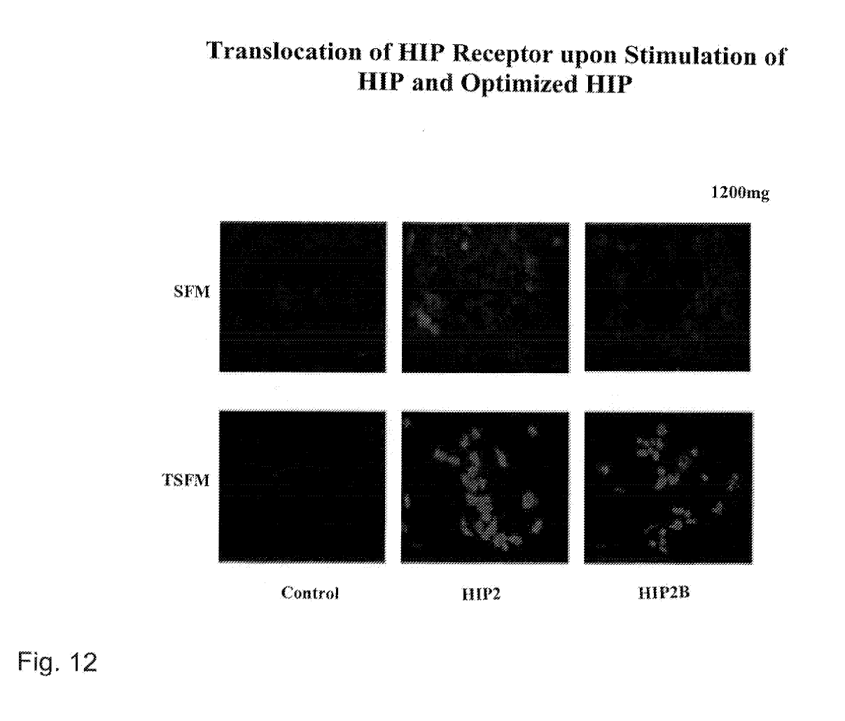

FIG. 12 demonstrates PANC-1 cells treated with trypsin and incubated in serum free medium differentiate into islet cell aggregates. HIP receptor/EXTL3 is upregulated during differentiation of human pancreatic cells and appears to interact with HIP2 and HIP2B interact with HIP receptor. HIP2 and HIP2B stimulated traffic from the HIP receptor on the cytoplasmic membrane into the nucleus of the cells stimulating differentiation of pancreatic progenitor cells into insulin-producing new islets.

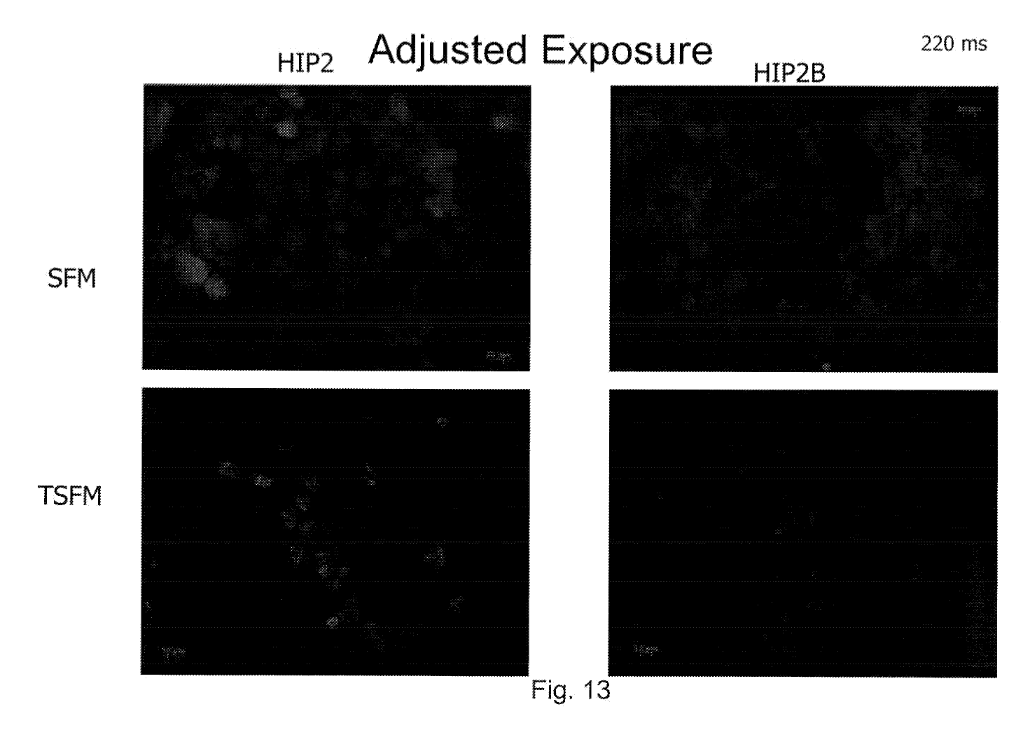

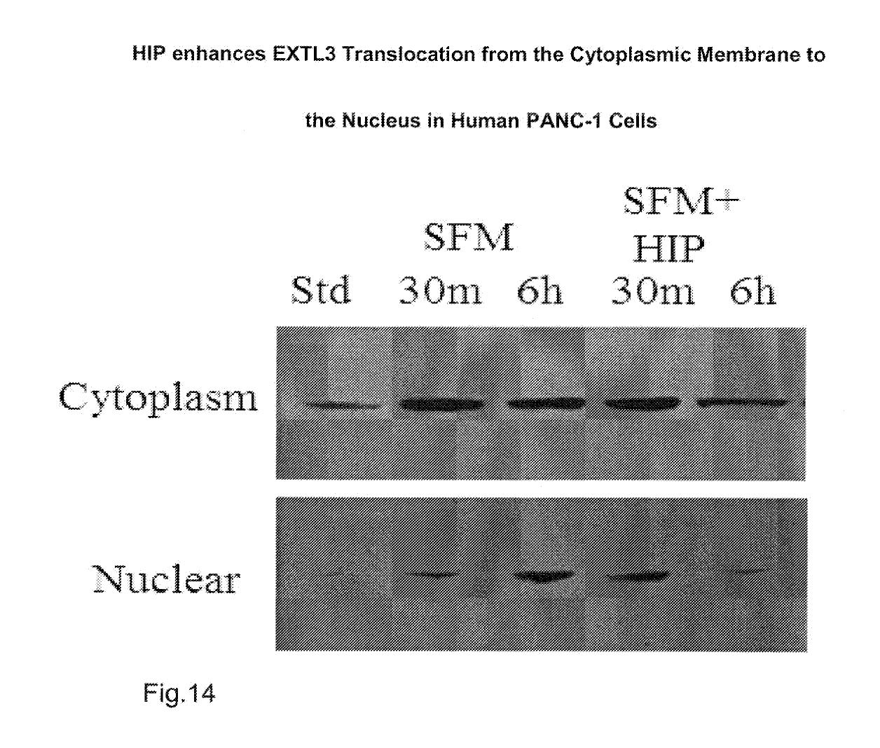

FIG. 13 demonstrates rabbit anti-human HIP receptor/EXTL3 antibody labeled with Cy3 in TSFM alone and TSFM with 15 μM (HIP) for 48 hours. HIP stimulates the receptor membrane bound protein to be engulfed by the cytoplasmic membrane and transported to the nuclear membrane under adjusted exposure.

### EXAMPLE 9

Western blot and immunofluorescence analyses of EXTL3 localization were performed in human PANC-1 cells grown under conditions that differentiate these cells into islet-like cells. EXTL3 is predominantly located on the external surface of the cytoplasmic membrane and its structure includes a single transmembrane region located inside of the cytoplasmic membrane. PANC-1 cells were grown in a standard growth medium or in a serum free medium (SFM) containing HIP.

FIG. 14 demonstrates the western blot and immunofluorescence analyses of EXTL3 localization in the PANC-1 cells revealed that when HIP was added to PANC-1 cells grown in SFM, EXTL3 appeared to translocate from the plasma membrane to the nucleus Western blot analyses of EXTL3 levels in cytosolic and nuclear fractions were isolated. Western blot analyses demonstrated that nuclear levels of EXTL3 were observed at 6 hours after culture in SFM without HIP. Inclusion of HIP in the culture media enhanced EXTL3 nuclear translocation at 30 minutes. These comparisons demonstrate that in the presence of HIP, there is accelerated translocation of EXTL3 from the cytoplasmic compartment to the nucleus and that EXTL3 nuclear translocation can be modulated in the presence of HIP.

### EXAMPLE 10

Using an antibody directed against human EXTL3, PANC-1 was immunostained and compared cells grown in SFM in the presence or absence of HIP. In standard medium, the cell borders were well-defined indicating surface expression of EXTL3 on the plasma membrane with undetectable levels of EXTL3 in the nucleus (FIG. 15). FIG. 15 demonstrates the immunofluorescent analyses of EXTL3 are shown in the upper panels indicated by the Cy3 immunofluorescent staining of EXTL3 (red). In the lower panel of images the Cy3 immunostaining of EXTL3 has been overlaid with DAPI (blue) staining of the nuclei. Cells were grown in standard growth medium as a control and compared to cells grown serum-free medium (SFM) in the presence or absence of HIP. The yellow arrows demonstrate examples of the surface expression of EXTL3 grown in standard growth medium. The cell borders are well-defined indicating surface expression of EXTL3 on the plasma membrane. The yellow arrows delineate the cell borders while the nuclei are shown in blue. The middle images are cells grown in SFM. EXTL3 is localized in the cytoplasm as indicated by cytoplasmic Cy3 staining. The green arrows show the lack of staining in the position of the nuclei. The green arrows in the lower image of cells grown in SFM demonstrate intense blue DAPI staining of the nuclei indicating a lack of EXTL3 in the nucleus. In the upper image of the cells grown in SFM and HIP, the presence of EXTL3 immunostaining in the nucleus indicated by the blue arrows suggests a translocation of EXTL3 into the nucleus. In the lower image of cells grown in SFM and HIP, the blue arrows indicate the position of the nuclei. In the lower image there is an overlap of EXTL3-Cy3 staining and nuclear DAPI staining that corroborates the nuclear localization of EXTL3 (blue arrows). Scale bar=20 μm in all images. In cells grown in SFM and HIP, EXTL3 was present in both the cytoplasm and the nucleus as indicated by the overlap of Cy3 and DAPI staining, suggesting that HIP facilitated a translocation of EXTL3 into the nucleus.

### EXAMPLE 11

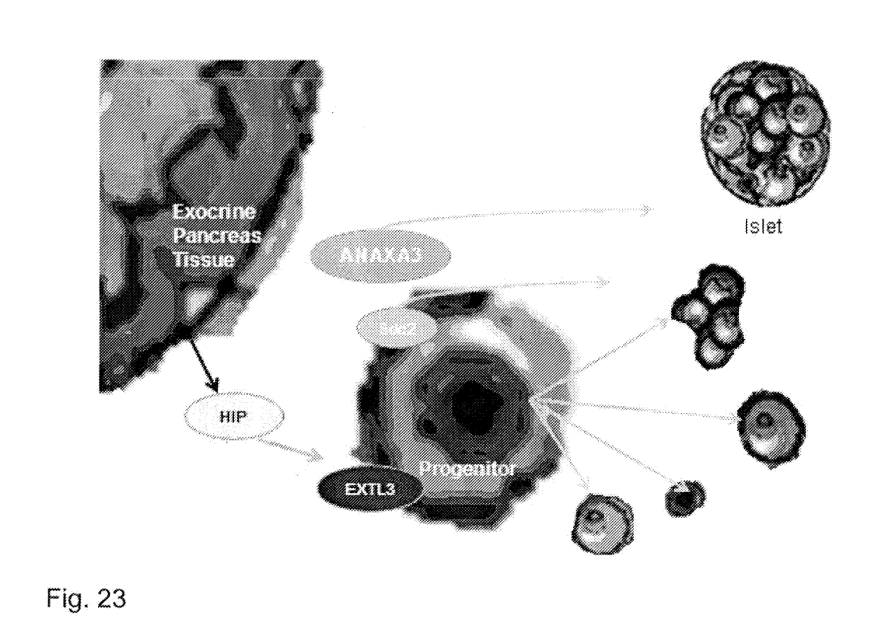

Without being bound by theory, FIG. 23 shows a proposed pathway of islet neogenesis in humans. Reg 3a is hypothesized to be the rate-limiting factor in this pathway and is secreted within the pancreas. Binding of the HIP region of Reg 3a initiates the interaction between HIP and EXTL3, leading to translocation of EXTL3 from the cytoplasmic membrane to the nucleus. This, in turn, triggers signaling leading to differentiation of the progenitor into the four endocrine cell types required for islet neogenesis. SDC2 and FN1 may participate in the process of islet genesis by aiding in the signaling of progenitor cell differentiation and ANXA3 may promote angiogenesis and innervation of developing islets.

Interacting proteins demonstrating interaction with of HIP with the HIP receptor, recruitment of Reg1b, PDX-1, PAX-1, NGN3 by HIP further coordinates and regulates endocrine islet function and beta cell proliferation are of particular use.

The results demonstrate that the HIP peptide derived from human REG 3a gene is sufficient to stimulate insulin production in human ductal pancreatic cells and restore glycemic control in mice with STZ-induced diabetes. The finding that HIP increased the number of islets in diabetic animals suggests that HIP restores glucose metabolism by stimulating the formation of new islets. Without being bound by theory, it is hypothesized that 1) newly formed islets provide a source of beta cells for subsequent beta cell expansion and proliferation 2) REG 3a may be the rate limiting or pacing component of islet neogenesis and 3) progenitor cells that are capable of differentiating into islets are present, most notably, in the ductal compartment of the adult pancreas.

Islet neogenesis is conventionally thought to occur almost exclusively in utero when the pancreas is being populated with islets for the first time, but also occurs during times of great pancreatic stress such as with acute pancreatitis or with a pancreatic stone. In contrast, beta cells within existing islets are capable of great plasticity during the postnatal period, undergoing changes in size and number depending on the insulin demands of an individual. Despite the low abundance of Reg 3a during the postnatal period, there is now evidence to suggest that new islet formation can occur in the adult through differentiation of pancreatic progenitor cells that are dispersed throughout the exocrine tissue, which comprises most of the pancreas.

The data suggest that the pathway for the differentiation of new islets is separate from that of beta regeneration within existing islets, but the two may be linked as evidenced by the capacity in adulthood to generate new islets in times of acute stress. Data suggests that HIP is involved in recruitment of a number of transcription factors including neurogenin 3, PDX 1 and PAX 1 required for the development of fully functional islets from progenitor cells and required for subsequent beta cell regeneration once new islets are formed. Thus, without being bound by theory, it is hypothesized that Syndecan 2 (SDC2), Fibronectin-1 (FN1) and Annexin3 (ANXA3) participate in the process of islet neogenesis by aiding in the signaling of progenitor cell differentiation and promoting angiogenesis and innervation of developing islets.

When new islets are generated, there is a nidus of beta cells for further replication based upon an individual's glucose milieu, insulin sensitivity and environmental and genetic factors. Diabetes arises when the beta cells can no longer respond to the demand for insulin. There is also accelerated apoptosis as the beta cells are pushed to exhaustion. Thus, in vivo generation of new islets via islet neogenesis from one's own progenitor cells may serve as novel approach to future therapies for diabetes.

Although EXTL3 functions in glycan polymer biosynthesis, it is also a component of the HIP signaling pathway. EXTL3 is unique amongst the exostoses proteins in that is has a transmembrane region, which likely confers its membrane localization and ability to act as a receptor. The in vitro data suggests that HIP may be necessary to initiate an interaction with EXTL3 in human pancreas, which may be the initiating step in signal transduction leading to islet neogenesis. Additional studies will be necessary to clarify the role of EXTL3 in HIP-induced islet neogenesis. Furthermore, identifying the other proteins activated by this peptide should uncover the signaling pathways responsible for islet neogenesis and may lead to new strategies for the treatment of type 1 and type 2 diabetes.

As disclosed herein, HIP binds to a specific receptor. The interaction of HIP with the HIP receptor in the presence of other proteins stimulates a signal pathway leading to islet neogenesis and may be used in the treatment of diabetes and related metabolic disorders.

Although the present invention has been described in considerable detail with reference to certain preferred embodiments thereof, other versions are possible. Therefore, the spirit and scope of the appended claims should not be limited to the description and the embodiments disclosed herein.

1. A peptide capable of binding to the HIP receptor.

2. The peptide of claim 1, wherein the peptide is a peptidomimetic.

3. The peptide of claim 2, wherein the peptide comprises an amino acid sequence selected from the group consisting of SEQ ID NO: 3 or SEQ ID NO: 6.

4. The peptide of claim 3, wherein the peptidomimetic has at least 85% amino acid sequence identity to SEQ ID NO: 3 or SEQ ID NO: 6.

5. The peptide of claim 3, wherein the peptidomimetic has at least 90% amino acid sequence identity to SEQ ID NO: 3 or SEQ ID NO: 6.

6. The peptide of claim 3, wherein the peptidomimetic has at least 95% amino acid sequence identity to SEQ ID NO: 3 or SEQ ID NO: 6.

7. A method of identifying a compound that binds to the HIP receptor comprising:
providing a HIP receptor and a cognate ligand of the HIP receptor;
combining the cognate ligand and the HIP receptor in the presence of a test compound under conditions wherein, in the absence of the test compound, a pre-determined quantity of the HIP peptide would bind the HIP receptor; and
determining if the quantity of the HIP peptide bound to the HIP receptor is decreased in the presence of the test compound, the decrease being indicative that the test compound binds the HIP receptor.

8. The method of claim 7, wherein the cognate ligand is a peptide.

9. The method of claim 8, wherein the peptide is a peptidomimetic.

10. The method of claim 9, wherein the peptidomimetic comprises an amino acid sequence selected from the group consisting of SEQ ID NO: 3 and SEQ ID NO: 6.

11. The method of claim 10, wherein the peptidomimetic has at least 85% amino acid sequence identity to SEQ ID NO: 3 or SEQ ID NO: 6.

12. The method of claim 10, wherein the peptidomimetic has at least 90% amino acid sequence identity to SEQ ID NO: 3 or SEQ ID NO: 6.

13. The method of claim 10, wherein the peptidomimetic has at least 95% amino acid sequence identity to SEQ ID NO: 3 or SEQ ID NO: 6.

14. The method of claim 10, wherein the test compound is a small organic molecule or a peptide.

15. The method of claim 10, wherein the test compound is a HIP receptor agonist.

16. The method of claim 10, wherein the test compound is a HIP receptor antagonist.

17. The method of claim 15, wherein the receptor agonist stimulates islet neogenesis.

18. A method of treating disease associated with aberrant glucose, wherein the disease is selected from the group consisting of: type 1 or type 2 diabetes, hyperglycemia, insulin resistant syndrome, metabolic syndrome, obesity, polycystic ovarian syndrome (PCOS), fasting hyperlipidemia/hypercholesterolemia, elevated fasting total cholesterol, elevated LDL and VLDL cholesterol, impotence, sexual dysfunction, and related conditions.

19. The method of claim 18, wherein the method of treatment comprises administration of an effective amount of a islet cell growth modulator selected from the group consisting of Reg 1a, syndecan 2, fibronectin 1, annexin A3, Reg1b, PDX-1, PAX-1, NGN3, peptidomimetics thereof, and ligands thereof.

20. The method of claim 19, wherein the cognate ligand is an agonist of the regulatory protein.

21. The method of claim 19, wherein the cognate ligand is an antagonist of the regulatory protein.


SEQUENCE LISTING (38 sequences)

SEQ ID NO: 1 - protein, 15 aa, Mesocricetus auratus
IGLHDPSHGTLPNGS

SEQ ID NO: 2 - protein, 15 aa, Homo sapiens
IGLHDPTQGTEPNGE

SEQ ID NO: 3 - protein, 15 aa, Homo sapiens
WIGLHDPTQGTEPNG

SEQ ID NO: 4 - protein, 14 aa, Homo sapiens
IGLHDPTQGTEPNG

SEQ ID NO: 5 - protein, 15 aa, Homo sapiens
  SITE (1)..(1): ACYLATION
IGLHDPTQGTEPNGE

SEQ ID NO: 6 - protein, 15 aa, Homo sapiens
  SITE (1)..(1): ACYLATION
WIGLHDPTQGTEPNG

SEQ ID NO: 7 - protein, 14 aa, Homo sapiens
  SITE (1)..(1): ACYLATION
IGLHDPTQGTEPNG

SEQ ID NO: 8 - protein, 16 aa, Homo sapiens
IGLHDPTQGTEPNGEC

SEQ ID NO: 9 - protein, 16 aa, Homo sapiens
TIGLHDPTQGTEPNGC

SEQ ID NO: 10 - protein, 15 aa, Homo sapiens
IGLHDPTQGTEPNGC

SEQ ID NO: 11 - protein, 30 aa, Homo sapiens
IGLHDPTQGTEPNGCCGNPETGQTPDHLGI

SEQ ID NO: 12 - protein, 34 aa, Homo sapiens
WIGLHDPTQGTEPNGECCEGNPETGQTPDHLGIW

SEQ ID NO: 13 - protein, 30 aa, Homo sapiens; identical to SEQ ID NO: 11

SEQ ID NO: 14 - protein, 16 aa, Homo sapiens
  SITE (1)..(1): ACYLATION
IGLHDPTQGTEPNGEC

SEQ ID NO: 15 - protein, 16 aa, Homo sapiens
  SITE (1)..(1): ACYLATION
WIGLHDPTQGTEPNGC

SEQ ID NO: 16 - protein, 15 aa, Homo sapiens
  SITE (1)..(1): ACYLATION
IGLHDPTQGTEPNGC

SEQ ID NO: 17 - protein, 16 aa, Homo sapiens
  SITE (1)..(1): ACYLATION
IGLHDPTQGTEPNGEC

SEQ ID NO: 18 - protein, 16 aa, Homo sapiens
  SITE (1)..(1): ACYLATION
WIGLHDPTQGTEPNGC

SEQ ID NO: 19 - protein, 15 aa, Homo sapiens
  SITE (1)..(1): ACYLATION
IGLHDPTQGTEPNGC

SEQ ID NO: 20 - protein, 16 aa, Homo sapiens
  SITE (16)..(16): PEGYLATION
IGLHDPTQGTEPNGEC

SEQ ID NO: 21 - protein, 16 aa, Homo sapiens
  SITE (16)..(16): PEGYLATION
WIGLHDPTQGTEPNGC

SEQ ID NO: 22 - protein, 15 aa, Homo sapiens
  SITE (15)..(15): PEGYLATION
IGLHDPTQGTEPNGC

SEQ ID NO: 23 - protein, 16 aa, Homo sapiens
  SITE (1)..(1): ACYLATION
IGLHDPTQGTEPNGEC

SEQ ID NO: 24 - protein, 16 aa, Homo sapiens
  SITE (1)..(1): ACYLATION
WIGLHDPTQGTEPNGC

SEQ ID NO: 25 - protein, 15 aa, Homo sapiens
  SITE (1)..(1): ACYLATION
IGLHDPTQGTEPNGC

SEQ ID NO: 26 - protein, 175 aa, Homo sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 27 - protein, 166 aa, Homo sapiens
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 28 - protein, 14 aa, Homo sapiens
IGLHDPTQGTEPNG

SEQ ID NO: 29 - protein, 15 aa, Mesocricetus auratus
IGLHDPSHGTLPNGS

SEQ ID NO: 30 - protein, 16 aa, Homo sapiens
WIGLHDPTQGTEPNGE

SEQ ID NO: 31 - protein, 16 aa, Pan troglodytes
WIGLHDPTQGSEPDGG

SEQ ID NO: 32 - protein, 16 aa, Mesocricetus auratus
WIGLHDPSHGTLPNGS

SEQ ID NO: 33 - protein, 16 aa, Mus musculus
WIGLHDPTMGQQPNGG

SEQ ID NO: 34 - protein, 16 aa, Rattus norvegicus
WIWLHDPTMGQQPNGG

SEQ ID NO: 35 - protein, 16 aa, Bos taurus
WIGLHDPTEGSEPDAG

SEQ ID NO: 36 - protein, 16 aa, Canis familiaris
WMGLHDPTEGYEPNAD

SEQ ID NO: 37 - protein, 16 aa, Ovis aries
WIGLHDPTEGSEPNAG

SEQ ID NO: 38 - protein, 919 aa, Homo sapiens
MTGYTMLRNGGAGNGGQTCMLRWSNRIRLTWLSFTLFVILVFFPLIAHYYLTTLDEADEAGKRIFGPRVGNELCEVKHVLDLCRIRESVSEELLQLEAKRQELNSEIAKLNLKIEACKKSIENAKQDLLQLKNVISQTEHSYKELMAQNQPKLSLPIRLLPEKDDAGLPPPKATRGCRLHNCFDYSRCPLTSGFPVYVYDSDQFVFGSYLDPLVKQAFQATARANVYVTENADIACLYVILVGEMQEPVVLRPAELEKQLYSLPHWRTDGHNHVIINLSRKSDTQNLLYNVSTGRAMVAQSTFYTVQYRPGFDLVVSPLVHAMSEPNFMEIPPQVPVKRKYLFTFQGEKIESLRSSLQEARSFEEEMEGDPPADYDDRIIATLKAVQDSKLDQVLVEFTCKNQPKPSLPTEWALCGEREDRLELLKLSTFALIITPGDPRLVISSGCATRLFEALEVGAVPVVLGEQVQLPYQDMLQWNEAALVVPKPRVTEVHFLLRSLSDSDLLAMRRQGRFLWETYFSTADSIFNTVLAMIRTRIQIPAAPIREEAAAEIPHRSGKAAGTDPNMADNGDLDLGPVETEPPYASPRYLRNFTLTVTDFYRSWNCAPGPFHLFPHTPFDPVLPSEAKFLGSGTGFRPIGGGAGGSGKEFQAALGGNVPREQFTVVMLTYEREEVLMNSLERLNGLPYLNKVVVVWNSPKLPSEDLLWPDIGVPIMVVRTEKNSLNNRFLPWNEIETEAILSIDDDAHLRHDEIMFGFRVWREARDRIVGFPGRYHAWDIPHQSWLYNSNYSCELSMVLTGAAFFHKYYAYLYSYVMPQAIRDMVDEYINCEDIAMNFLVSHITRKPPIKVTSRWTFRCPGCPQALSHDDSHFHERHKCINFFVKVYGYMPLLYTQFRVDSVLFKTRLPHDKTKCFKFI